(19) United States
(12) Patent Application Publication
(10) Pub. No.: US 2004/0213782 A1
(43) Pub. Date: Oct. 28, 2004

(54) Compositions of an aquaporin modulating agent and an aqueous humor modulating agent for the treatment of elevated intraocular pressure

(75) Inventor: Martin B. Wax, Westlake, TX (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/768,266

(22) Filed: Jan. 30, 2004

Related U.S. Application Data
(60) Provisional application No. 60/444,509, filed on Feb. 3, 2003.

(51) Int. Cl. A61K 39/395; A61K 31/225; A61K 31/401

(52) U.S. Cl. 424/143.1; 514/423; 514/548

(57) ABSTRACT

The present invention provides compositions and methods for lowering intraocular pressure in a subject. More particularly, the invention provides a combination therapy for the treatment of an ophthalmic disorder mediated by an elevated intraocular pressure comprising administering to a subject an aquaporin modulating agent in combination with an aqueous humor modulating agent, where the aqueous humor modulating agent lowers intraocular pressure by a pathway other than the modulation of aquaporin.

### CROSS REFERENCE TO RELATED APPLICATION

This application claims priority from Provisional Application Serial No. 60/444,509 filed on Feb. 3, 2003, which is hereby incorporated by reference in its entirety.

### FIELD OF THE INVENTION

The present invention provides compositions and methods for lowering intraocular pressure. More particularly, the invention is directed toward a combination therapy for the treatment of an ophthalmic disorder mediated by elevated intraocular pressure comprising administering to a subject an aquaporin modulating agent in combination with an aqueous humor modulating agent.

### BACKGROUND OF THE INVENTION

The continued increase in the incidence of ophthalmic disorders mediated by elevated intraocular pressure (IOP), including glaucoma, provides compelling evidence that there is a continuing need for better treatment strategies. Glaucoma, for example, is consistently among the leading causes of blindness and optic nerve damage among adults in the United States. Generally speaking, glaucoma is characterized by a progressive neuropathy caused in part by deleterious effects resulting from increased IOP on the optic nerve. In normal individuals, IOPs range from 12 to 20 mm Hg., averaging approximately 16 mm Hg. But in individuals suffering from glaucoma, IOPs typically rise to 25 mm Hg. or greater, and can sometimes exceed 40 mm Hg resulting in rapid and permanent visual loss. Loss of vision can result from IOPs only slightly above the normal range in eyes that are unusually pressure-sensitive over a period of years. Moreover, extremely high pressures, e.g., 70 mm Hg., may cause blindness within only a few days if left untreated.

Two mainstays of glaucoma treatment are decreasing aqueous humor production, or enhancing its outflow from the eye. Aqueous humor is the fluid that fills the chamber of the eye behind the cornea and in front of the lens. It is formed through the ciliary body, and is secreted constantly into the posterior chamber resulting in a continual flow between the iris and the lens and through the pupil into the chamber of the eye. In individuals with an IOP in the normal range, aqueous humor concentration is maintained as a delicate equilibrium mediated by the balance between its production and outflow. When everything functions correctly, ocular pressure is normal and aqueous humor inflow is approximately equal to outflow. But when this equilibrium is disrupted by factors such as aging, inflammation, hemorrhage, or cataracts, IOP may become dangerously elevated if left untreated.

All therapies currently employed to treat ophthalmic disorders mediated by elevated IOP are restricted to reducing IOP by either affecting the production or outflow of aqueous humor. Depending upon the type and severity of the condition, either surgical or pharmacological treatments may be employed to lower IOP. By way of example, both laser and incisional surgical procedures may be used for the treatment of severe conditions such as open-angle glaucoma. Angle-closure glaucoma entails closure or blockage of the anterior chamber angle, thereby restricting outflow of aqueous humor. While pharmacological agents generally effectively control mild cases of open-angle glaucoma, laser trabeculoplasty or filtering surgery to improve aqueous drainage is employed in severe cases. Though often necessary and quite effective for many types of glaucoma, surgical intervention is an invasive form of treatment, even if local anesthesia can be used.

Several classes of pharmacological agents may also be employed to lower IOP. One such class of pharmacological agent is miotic agents. Though their precise mechanism of action has not yet been fully elucidated, miotic drugs lower IOP by facilitating aqueous humor outflow. Mydriatic agents are also useful for lowering IOP. For example, the sympathomimetic amines, such as epinephrine and dipivefrin, lower IOP, at least in part through stimulation of beta₂-adrenergic receptors in the trabecular meshwork. Additionally, alpha₂-adrenergic agonists (e.g. apraclonidine) have been shown to be effective in lowering IOP by inhibition of aqueous humor formation. Moreover, both non-selective beta₁- and beta₂-adrenergic blocking agents (e.g., timolol and levobunolol) and beta₁-selective (e.g., betaxolol) adrenergic blocking agents are also used to lower IOP. Prostaglandin compounds have also been shown to have an ocular hypotensive activity. Although these pharmacological agents are all less invasive than surgical intervention, they never-the-less are still often accompanied by adverse effects (e.g. conjunctival irritation, burred vision, ocular pain, and headaches) at the dosages required for effective treatment.

Aquaporins (AQP), a large family of membrane proteins that function as highly selective water channels, have also been identified as a target for modulating IOP. At least ten AQPs, numbered 0 through 9, have been identified from various mammalian tissues (e.g. brain, kidney, salivary gland, testis, and liver) and AQPs 0 through 5 have been identified in the eye. Several studies have described functional roles for AQPs in ocular physiology. For example, inhibition of AQP1 using antisense oligonucleotides reduces the fluid movement across the ciliary epithelial cells in culture (Hamann et al., (1998) Am. J. Physiol. 274:C1332-1345); and mutations in AQP0 result in congenital cataracts (Shiels, A. and Bassnett, S. (1996) Nature Genet 12:212-215). It was also shown that AQP1-knockout mice have lower IOP and aqueous humor production (Zhang et al., (2002) J. Gen Physiol 119:561-569).

### SUMMARY OF THE INVENTION

Among the aspects of the present invention is provided a method for lowering IOP in a subject comprising administering to the subject an aquaporin modulating agent in combination with an aqueous humor modulating agent, where the aqueous humor modulating agent lowers intraocular pressure by a pathway other than the modulation of aquaporin.

Another aspect of the invention provides a method to treat an ophthalmic disorder mediated by an elevated IOP in a subject comprising administering to the subject an aquaporin modulating agent and an aqueous humor modulating agent, where the aqueous humor modulating agent lowers intraocular pressure by a pathway other than the modulation of aquaporin. In one embodiment, the ophthalmic disorder is a glaucoma disorder. In one alternative of this embodiment, the glaucoma disorder is primary angle closure glaucoma. In another alternative of this embodiment, the glaucoma disorder is secondary open angle glaucoma. In another embodiment, the ophthalmic disorder is ocular hypertension.

In still another aspect of the invention is provided a method to treat a glaucoma disorder in a subject comprising administering to the subject an aquaporin modulating agent and an aqueous humor modulating agent. In one embodiment, the glaucoma disorder is primary angle closure glaucoma. In another embodiment, the glaucoma disorder is secondary open angle glaucoma.

A further aspect of the invention provides a composition comprising an aquaporin modulating agent and an aqueous humor modulating agent.

In one embodiment, the aquaporin modulating agent alters the expression of aquaporin. In another embodiment, the agent alters expression by substantially inhibiting aquaporin gene expression. In one alternative of this embodiment, the aquaporin expression inhibitor is a carbonic anhydrase inhibitor, vasopressin, or an angiotensin converting enzyme inhibitor. In another alternative of this embodiment, the aquaporin expression inhibitor is an aquaporin antisense oligonucleotide or a ribozyme.

In another embodiment, the aquaporin modulating agent inhibits or enhances the function of aquaporin. In one alternative of this embodiment, the aquaporin modulating agent is a protein kinase C activator. In another alternative of this embodiment, the aquaporin modulating agent is a protein kinase A inhibitor.

In yet another embodiment, the aqueous humor modulating agent is a prostaglandin, a beta adrenergic antagonist blocker, an adrenergic agonist, a cholinergic agonist, or a carbonic anhydrase inhibitor.

Other aspects and features of the invention are described in more detail below.

### DEFINITIONS AND ABBREVIATIONS

The term “subject” for purposes of treatment includes any human or animal subject who is susceptible to an elevated IOP. The subject can be a domestic livestock species, a laboratory animal species, a zoo animal or a companion animal. In one embodiment, the subject is a mammal. In another embodiment, the mammal is a human being.

The phrase “therapeutically-effective” is intended to qualify the amount of each agent (i.e. the amount of AQP modulating agent and the amount of aqueous humor modulating agent) that will achieve the goal of improvement in disorder severity and the frequency of incidence over no treatment or treatment of each agent by itself.

### DESCRIPTION OF THE PREFERRED EMBODIMENTS

The present invention provides a combination therapy comprising the administration to a subject of a therapeutically effective amount of an AQP modulating agent in combination with a therapeutically effective amount of an aqueous humor modulating agent, where the aqueous humor modulating agent lowers intraocular pressure by a pathway other than the modulation of aquaporin. The combination therapy is used to lower IOP, and to treat ophthalmic disorders mediated by elevated IOP. When administered as part of a combination therapy, the AQP modulating agent together with the aqueous humor modulating agent provide enhanced treatment options as compared to administration of either the AQP modulating agent or the aqueous humor modulating agent alone.

Aquaporin Modulating Agents

In general, one aspect of the present invention is the use of an AQP modulating agent that lowers IOP. Typically, the agent selected will lower IOP by reducing the production of aqueous humor via either modulating the expression of AQP or by modulating its function once expressed. The agent may also lower IOP by modifying the secretion of aqueous humor from the eye once it is produced. By way of example, the agent selected may lower IOP by increasing the outflow of aqueous humor from the anterior chamber of the eye. Moreover, the agent selected may be effective in modulating any of the various AQP isoforms, including AQP0 through AQP9, to the extent that modulating the isoform lowers IOP. Because of their prevalence in the eye, however, typically the agent will modulate one or more of AQP0 through AQP5 and more typically, the agent will modulate AQP1 or AQP4.

In one embodiment, the AQP modulating agent is tetraethylammonium or a pharmaceutically acceptable salt having the structure:

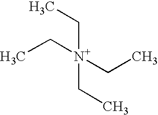

In another embodiment, the AQP modulating agent is any such agent described in WO 01/64219 A2, which is hereby incorporated by reference in its entirety. In one alternative of this embodiment, the AQP modulating agent is nocodazole or a pharmaceutically acceptable salt having the structure:

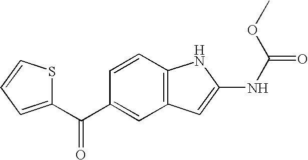

In still another alternative of this embodiment, the AQP modulating agent is a vinca alkyloid. For example, suitable vinca alkyloids include vincristine, vinblasine, and vinorelbine. In a further alternative of this embodiment, the AQP modulating agent is selected from the group consisting of colchicine, rhizoxin, estramustine, erbuluzole, tubulozole, and cytochalasin D.

Another aspect of the invention encompasses AQP modulating agents that lower IOP by altering the expression of an AQP gene. In some aspects, the agent may cause a decrease in the overall rate of AQP gene expression and concomitantly, result in a decrease in mature AQP. In other aspects, the agent may cause an increase in the overall rate of AQP gene expression. Likewise, the agent may modify expression of an AQP gene such that the amount of functional AQP decreases or increases. By way of example, the agent may cause premature termination of AQP gene transcription, thereby resulting in a shorter transcription product. By way of further example, the agent may alter or interrupt the sequence of the transcription product such that proper post transcription processing and translation of a functional AQP does not occur or occurs at a substantially reduced rate.

In one embodiment, the AQP modulating agent is an AQP antisense oligonucleotide. These agents are typically unmodified or modified antisense oligonucleotides directed against various AQP nucleic acid sequences that inhibit AQP gene transcription in both a sequence-specific and in a non-sequence specific manner. Because of their complementary, the agent binds to the AQP nucleic acid and thereby prevents its transcription. Of course, the particular antisense oligonucleotides employed will vary considerably depending upon its intended target within the AQP gene and one skilled in the art can readily design appropriate antisense oligonucleotides for use in the present invention. Methods for selecting and constructing antisense oligonucleotides suitable for use in the invention are more fully described, for example, in Hamann et al., (1998) Am. J. Physiol. 274:C1332-1345.

In yet another embodiment, the AQP modulating agent is a ribozyme. Ribozymes are RNA molecules having an enzymatic activity that are able to repeatedly cleave other separate RNA molecules in a nucleotide base sequence specific manner. Within the context of the present invention, the ribozyme employed typically cleaves AQP expressed RNA and in particular, mRNA targets, resulting in the destruction of mRNA transcript integrity. By way of example, the ribozyme employed may be targeted to and prevents the translation of mRNA encoding a region of AQP required for proper translation or translocation. By way of further example, the ribozyme employed may be targeted to and prevents the translation of mRNA encoding a region of AQP required for proper function of the mature protein.

In still another embodiment, the AQP modulating agent is a carbonic anhydrase (CA) inhibitor. A number of different CA inhibitors capable of lowering IOP by altering the expression of an AQP gene may be employed. Generally speaking, the CA inhibitor may inhibit any isomer of the metalloprotein enzyme that catalyzes the interconversion of CO₂ and H₂CO₃ (CO₂+O₂→HCO₂⁻+H⁺). Typically, however, the CA inhibitor will inhibit either the CAIV or CAIV isoform. By way of example, the CA inhibitor acetazolamide results in a significant decrease in the level of AQP1 expression in the epididymis of rats (Yu et al., (2002) Arch Androl 48(4):281-294). Other suitable CA inhibitors include methazolamide, dorzolamide hydrochloride ophthalmic solution, dorzolamide hydrochloride-timolol maleate ophthalmic solution, brinzolamide hydrochloride, dorzolamide, and brinzolamide.

In a further embodiment, the AQP modulating agent is an angiotensin converting enzyme inhibitor. A number of angiotensin converting enzyme inhibitors capable of lowering IOP by altering the expression of an AQP gene may be utilized. By way of example, angiotensin II increases the expression of AQP2 in the kidney of cardiomyopathic hamsters (Wong N L, and Tsui J K, (2002) Metabolism 51(8):970-975). Administration of the angiotensin converting enzyme inhibitor enalapril to the cardiomyopathic hamsters causes a significant decrease in the level of AQP2 expression so that it is comparable to the level of AQP2 expressed in normal hamsters (i.e. hamster that are not cardiomyopathic). Other angiotensin converting enzyme inhibitors suitable for use in the present invention include benazepril, captopril, fosinopril, lisinopril, moexipril, quinapril, ramipril, and trandolapril.

Yet another aspect of the invention encompasses AQP modulating agents that substantially alter the function of AQP. In some aspects, the agent may disrupt the ability of AQP to form a fluid membrane channel. For example, the agent may prevent proper assembly of AQP subunits such that AQP cannot embed within the plasma membrane and form a channel. Likewise, the agent may disrupt the ability of AQP to function as a fluid membrane channel. By way of example, the agent may bind to an AQP of a functional membrane channel and either permanently or transiently prevent the ability of fluid to pass through the channel. In other aspects, the agent may prevent the ability of AQP to form a gated ion channel, such as a cyclic GMP gated ion channel. By way of example, the agent may prevent phosphorylation of AQP at a site necessary for its ability to function as a gated ion channel. By way of further example, the agent may inactivate an intermediary compound necessary for AQP function.

In one embodiment, the AQP modulating agent is a protein kinase C (PKC) activator. PKC is a member of the protein kinase family responsible for regulating pathways of intermediary metabolism (e.g. glycogen phosphorylase kinase). Typically, when a PKC activating agent is employed, the AQP target is generally AQP4 (see e.g. Han et al., (1998) J. Biol. Chem. 273:6001-6004, demonstrating that the water channel activity of AQP4 is in part regulated by protein phosphorylation via a PKC pathway). Within the context of the invention, a number of agents that result in the activation of PKC may be employed. In one regulatory pathway, activation of PKC occurs when plasma membrane receptors coupled to phospholipase C are themselves activated causing the release of diacylglycerol, which in turn activates PKC. Generally speaking, the agent will typically be a diacylglycerol mimic that can directly activate PKC. In one aspect of this embodiment, the diacylglycerol mimic is a phorbol ester. Phorbol esters suitable for use in the present invention include phorbol 12, 13 dibutyrate, phorbol 12-myristate-12-acetate, phorbol 12-O-tetradecanoylphorbol 13-acetate, phorbol 12, 13 didecanoate and tetradecanoylphorbol acetate. In other aspects of this embodiment, the agent employed may indirectly activate PKC by activating phospholipase C causing the release of diacylglycerol. Likewise, the agent may activate PKC by a pathway that is independent from the diacylglycerol pathway. By way of example, ionomycin is a molecule that carries calcium through the plasma membrane to increase the calcium concentration in the cytoplasm and activate PKC without activating phospholipase C.

In another embodiment, the AQP modulating agent is an adenylate cyclase inhibitor. Adenylate cyclase is a membrane bound enzyme that converts adenosine triphosphate (ATP) to 3′, 5′-cyclic adenosine monophosphate (cAMP), which is a potent intracellular messenger. Accordingly, inhibition of adenylate cyclase concomitantly causes a reduction in intracellular cAMP levels. Typically, when an adenylate cyclase inhibitor is utilized, the AQP target is generally AQP1 (see e.g. Patil et al., (1997) Science 275:1492, demonstrating that the water channel activity of AQP1 is in part regulated by atrial natriuetic peptide, a known adenylate cyclase inhibitor). Within the context of the invention, a number of agents that result in the inhibition of adenylate cyclase may be employed. In one aspect of this embodiment, the agent is a natriuretic peptide that inhibits adenylate cyclase. Natriuretic peptides are any of several proteins that stimulate natriuresis. By way of example, suitable natriuretic peptides for use in the present invention include atrial natriuretic peptide (ANP) and brain natriuretic peptide (BNP).

In yet another embodiment, the AQP modulating agent inhibits a cAMP dependent protein kinase such as protein kinase A (PKA). PKA belongs to a class of protein kinases that are regulated by cAMP. Typically, when a PKA inhibitor is employed, the AQP target is generally AQP1 (see e.g. Yoo et al., (1996) Science 273(5279) 1216-1218, demonstrating that the water channel activity of AQP1 is in part regulated by a cAMP dependent mechanism via a PKA pathway). Within the context of the invention, a number of agents that result in the inhibition of PKA may be employed. Examples of suitable PKA inhibitors include (5-isoquinolinesulfonyl)piperazine; 1-(5-Isoquinolinesulfonyl)-2-methylpiperazine, 4-cyano-3-methylisoquinoline; adenosine 3′,5′-cyclic monophosphorothioate, 2′-O-monobutyryl; adenosine 3′,5′-cyclic monophosphorothioate; 8-bromo-2′-monobutyryl, adenosine 3′,5′-cyclic monophosphorothioate; 8-piperidino, N-(2-aminoethyl)-5-chloronaphthalene-1-sulfonamide; N-(2-aminoethyl)-5-isoquinolinesulfonamide; N-[2-((p-bromocinnamyl)amino)ethyl]-5-isoquinolinesulfonamide; N-(2-guanidinoethyl)-5-isoquinolinesulfonamide; 4,4′,5,5′,6,6′-hexahydroxydiphenic acid 2,6,2′,6′-dilactone; (5-isoquinolinesulfonyl)homopiperazine; N-[2-(methylamino)ethyl]-5-isoquinolinesulfonamide; and trans-3,3′,4,5′-tetrahydroxystilbene.

In still a further embodiment, the AQP modulating agent is a vasoactive peptide. As a class, vasoactive peptides are typically peptides that affect the diameter of a blood vessel. Typically, when a vasoactive peptide is utilized, the AQP target is generally AQP1 (see e.g. Patil et al., (1997) Biochem. Biophys. Res. Comm. 238:392-396, demonstrating that the water channel activity of AQP1 is in part regulated by the vasoactive peptides atrial natriuetic peptide and arginine vasopressin). Within the context of the invention, a number of vasoactive peptides that result in an inhibition of any AQP function may be employed. In one alternative of this embodiment, the vasoactive peptide is a vassopressin, such as arginine vasopressin. In another aspect of the invention, the vasoactive peptide is a natriuretic peptide such as ANP or BNP.

Of course it will be apparent to a skilled artisan that a particular AQP modulating agent may modulate AQP by a number of different mechanism. For example, a specific AQP modulating agent may decrease the expression of AQP and substantially inhibit its function once expressed. In other aspects, the AQP modulating agent may not impact either the expression or function of AQP. It is contemplated that all AQP modulating agents that lower IOP are within the scope of the invention irrespective of their mechanism of action.

Aqueous Humor Modulating Agents

In addition to an AQP modulating agent, the composition also an aqueous humor modulating agent. A number of aqueous humor modulating agents may be employed to the extent that they lower IOP. In general, the aqueous humor modulating agent may lower IOP by causing a reduction in the formation of aqueous humor. The aqueous humor modulating agent may also lower IOP by increasing the outflow of aqueous humor from the anterior chamber of the eye. Moreover, the aqueous humor modulating agent may lower IOP by decreasing the inflow of aqueous humor from the anterior chamber of the eye. Irrespective of a particular aqueous humor modulating agent's mechanism of action, it typically lowers IOP by a pathway other than the modulation of AQP.

In one aspect, the aqueous humor modulating agent is a prostaglandin or a prostaglandin analog. Naturally occurring prostaglandins are C-20 unsaturated fatty acids. Typically, any prostagladin or prostaglandin analog capable of lowering IOP by altering the production, inflow or outflow of aqueous humor may be used in the composition. Suitable prostaglandins that may be employed in the composition include prostaglandin A, prostaglandin B, prostaglandin D, prostaglandin E, prostaglandin F or any combination thereof. Typically, the prostaglandin employed is prostaglandin F or a homolog of prostaglandin F such as PGF₂ₐ. By way of example, PGF₂ₐ is characterized by hydroxyl groups at the C₉ and C₁₁ positions on the alicyclic ring, a cis-double bond between C₅ and C₆, and a trans-double bond between C₁₃ and C₁₄. PGF₂ₐ has the following formula:

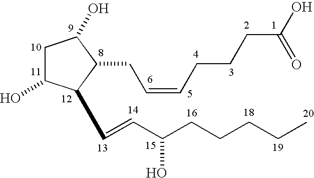

In another embodiment, the aqueous humor modulating agent is a prostaglandin analog. Typically, suitable prostaglandin analogs include any analogs that are similar in structure and function to prostaglandin, which lower IOP. In one alternative of this embodiment, the prostaglandin analog is a prostaglandin FP receptor antagonist. In another alternative of this embodiment, the prostaglandin analog is a prostaglandin F₂ₐ analog. In one embodiment, the prostaglandin F₂ₐ analog is lanaprost. In another embodiment, the F₂ₐ analog is travoprost. In still a further alternative of this embodiment, the prostaglandin analog is unoprostone. In a further alternative of this embodiment, the prostaglandin analog is a prostamide. Generally speaking, the prostamide employed may be any naturally occurring or synthetic prostamide. In one embodiment, the prostamide is the synthetic analog bimatoprost. The preparation and pharmaceutical profiles of several prostaglandin and prostaglandin analogs, including cloprostenol, fluprostenol, latanoprost, and travoprost, are more fully described in U.S. Pat. No. 5,510,383, which is hereby incorporated by reference in its entirety.

In a further aspect, the aqueous humor modulating agent is a beta adrenergic receptor antagonists. Beta adrenergic receptor antagonists bind beta-adrenergic receptors such as the beta₁ adrenergic receptor or the beta₂ adrenergic receptor. By binding to these receptors, the beta adrenergic receptor antagonists decrease the ability of the body's own natural epinephrine to bind to those receptors, leading to inhibition of various processes in the body's sympathetic system, including a reduction in aqueous humor secretion by ciliary tissues in the eye. Generally speaking, any beta adrenergic receptor antagonists capable of lowering IOP by altering the production, inflow or outflow of aqueous humor may be used in the composition. In some embodiments, the beta adrenergic receptor antagonists may be selective for the beta₁ adrenergic receptor. By way of example, suitable selective beta₁ adrenergic receptor antagonists include betaxolol and its enantiomer levobetaxolol. In other embodiments, the beta adrenergic receptor antagonists may be non-selective, blocking both the beta₁ adrenergic receptor and the beta₂ adrenergic receptor. Examples of suitable non-selective beta adrenergic receptor antagonists include timolol, levobunolol, carteolol and metipranolol.

In yet another aspect, the aqueous humor modulating agent is an adrenergic agonists. Adrenergic agonists typically bind to and stimulate adrenergic receptors, causing responses similar to those of adrenaline and noradrenaline, including the inhibition of aqueous humor production. In general, any adrenergic receptor agonists capable of lowering IOP by altering the production, inflow or outflow of aqueous humor may be used in the composition. In one embodiment, the adrenergic receptor agonist is alpha-2 adrenergic receptor agonists. By way of example, suitable alpha-2 adrenergic receptor agonists include apraclonidine and brimonidine. In a further embodiment, the adrenergic receptor agonist is epinephrine. In some embodiments, the adrenergic receptor agonists may be a pharmaceutically acceptable salt of epinephrine such as epinephryl borate, epinephrine bydrochloride or epinephrine bitartate. In other embodiments, the adrenergic receptor agonist may be a prodrug of epinephrine such as dipivefrin.

In still another aspect, the aqueous humor modulating agent is a mitotic. Generally speaking, miotics are divided into two categories: direct and indirect cholinergic agents. Irrespective of their classification, mitotic agents generally lower IOP by stimulating smooth muscle muscarinic receptors, causing a widening of the trabecular meshwork to increase aqueous humor outflow. By way of example, suitable direct cholinergic agents include pilocarpine, pilocarpine hydrochloride, and carbachol. Examples of suitable indirect cholinergic agents include echothiophate iodide, echothiophate, demacarium, and physostigmine.

In a further aspect, the aqueous humor modulating agent is a carbonic anhydrase inhibitor. CA is an enzyme involved in producing bicarbonate, which is required for aqueous humor production by the ciliary tissues in the eye. By inhibiting CA, accordingly, production of aqueous humor is substantially reduced. Generally speaking, the CA inhibitor may inhibit any isomer of the metalloprotein enzyme that catalyzes the interconversion of CO₂ and H₂CO₃ (CO₂+O₂→HCO₂⁻+H⁺). Typically, however, the CA inhibitor will inhibit the CAI, CAII or CAIV isoform. Examples of suitable CA inhibitors include acetazolamide, methazolamide, dorzolamide hydrochloride ophthalmic solution, dorzolamide hydrochloride-timolol maleate ophthalmic solution, brinzolamide hydrochloride, dorzolamide, and brinzolamide.

Other aqueous humor modulating agents that may be used to reduce IOP include cannabinoids drug class, for example, anandamine; selective and unselective PKC inhibitors drug class; rho kinase inhibitors drug class; and combinations thereof; corticosteroid receptor antagonists; selective and nonselective dopamine DA-1 agonists; TNF antagonists; somatostatin selective sst4 agonists; angiotensin II antagonists; thyroxine; adenosine 3 antagonists, vacuolar proton ATPase inhibitors such as bafilomycin; sodium hydrogen antiporter inhibitors; chloride anion exchanger inhibitors; and combinations thereof.

It is contemplated that the composition may include more than one aqueous humor modulating agent. Generally speaking, combinations are selected so as to include agents that have different modes of action and work on different receptor sites or enzymes, but that do not antagonize one another. By way of illustrative example, an ineffective combination may include brimonidine with a beta blocker and brimonidine with epinephrine. Both brimonidine and beta blockers suppress the formation of cAMP in the ciliary epithelium, while epinephrine upregulates the adenyl cyclase enzyme that brimonidine indirectly inhibits. By way of further illustrative example, an effective combination may include a beta blocker with a cholinergic agent or a beta blocker with a CA inhibitor, as both combinations include agents that target different receptor sites or enzymes.

Routes of Administration

Generally speaking, the AQP modulating agent and aqueous humor modulating agents useful in the practice of the present invention can be formulated into pharmaceutical compositions and administered separately, either simultaneously or sequentially. Alternatively, the AQP modulating agent and aqueous humor modulating agent can be formulated into a single composition comprising both agents. Irrespective of whether both agents are formulated into a single composition or formulated with each agent in a separate composition, the composition may be administered by any means that will deliver a therapeutically effective dose of both agents, as detailed herein or as otherwise known in the art. For example, formulation of agents is discussed in Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

In one aspect, the composition is administered directly to the eye by any means known in the art such as in a solution, cream, ointment, emulsion, suspension and slow release formulations. Administration of a composition to the eye generally results in direct contact of the agents with the cornea, through which at least a portion of the administered agents pass. In general, the composition has an effective residence time in the eye of about 2 to about 24 hours, more typically about 4 to about 24 hours and most typically about 6 to about 24 hours.

A composition of the invention can illustratively take the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition may include a gel formulation. In other embodiments, the liquid composition is aqueous. Alternatively, the composition can take the form of an ointment.

In one embodiment, the composition is an aqueous solution, suspension or solution/suspension, which can be presented in the form of eye drops. By means of a suitable dispenser, a desired dosage of each agent can be metered by administration of a known number of drops into the eye. For example, for a drop volume of 25 μl, administration of 1-6 drops will deliver 25-150 μl of the composition. Aqueous compositions of the invention typically contain from about 0.01% to about 50%, more typically about 0.1% to about 20%, still more typically about 0.2% to about 10%, and most typically about 0.5% to about 5%, weight/volume of the AQP modulating agent and aqueous humor modulating agent.

Generally speaking, aqueous compositions of the invention have ophthalmically acceptable pH and osmolality. “Ophthalmically acceptable” with respect to a formulation, composition or ingredient typically means having no persistent detrimental effect on the treated eye or the functioning thereof, or on the general health of the subject being treated. It will be recognized that transient effects such as minor irritation or a “stinging” sensation are common with topical ophthalmic administration of agents and the existence of such transient effects is not inconsistent with the formulation, composition or ingredient in question being “ophthalmically acceptable” as detailed herein. But formulations, compositions and ingredients employed in the present invention are those that generally cause no substantial detrimental effect, even of a transient nature.

In an aqueous suspension or solution/suspension composition, the agent can be present predominantly in the form of nanoparticles, i.e., solid particles smaller than about 1000 nm in their longest dimension. A benefit of this composition is more rapid release of the agent, and therefore more complete release during the residence time of the composition in a treated eye than occurs with larger particle size. Another benefit is reduced potential for eye irritation by comparison with larger particle size. Reduced eye irritation in turn leads to a reduced tendency for loss of the composition from the treated eye by lacrimation, which is stimulated by such irritation.

In a related composition, the agent typically has a D₉₀ particle size of about 10 to about 2000 nm, wherein about 25% to 100% by weight of the particles are nanoparticles. “D₉₀” is a linear measure of diameter having a value such that 90% by volume of particles in the composition, in the longest dimension of the particles, are smaller than that diameter. For practical purposes a determination of D₉₀ based on 90% by weight rather than by volume is generally suitable.

In one composition, substantially all of the agent particles in the composition are smaller than 100 nm, i.e., the percentage by weight of nanoparticles is 100% or close to 100%. Generally speaking, the average particle size of the agent in this embodiment is typically about 100 to about 800 nm, more typically about 150 to about 600 nm, and even more typically, about 200 to about 400 nm. The agent can be in crystalline or amorphous form in the nanoparticles. Processes for preparing nanoparticles that involve milling or grinding typically provide the agent in crystalline form, whereas processes that involve precipitation from solution typically provide the agent in amorphous form.

The ophthalmic composition in some embodiments can be an aqueous suspension of an agent of low water solubility, wherein typically the agent is present predominantly or substantially entirely in nanoparticulate form. Without being bound by theory, it is believed that release of the agent from nanoparticles is significantly faster than from a typical “micronized” composition having a D₉₀ particle size of, for example, about 10,000 nm or greater.

In another embodiment, an aqueous suspension composition of the invention can comprise a first portion of the agent in nanoparticulate form, to promote relatively rapid release, and a second portion of the agent having a D₉₀ particle size of about 10,000 nm or greater, that can provide a depot or reservoir of the agent in the treated eye for release over a period of time, for example about 2 to about 24 hours, more typically about 2 to about 12 hours, to promote sustained therapeutic effect and permit a reduced frequency of administration.

In still another embodiment, an aqueous suspension can contain one or more polymers as suspending agents. Useful polymers include water-soluble polymers such as cellulosic polymers, e.g., hydroxypropyl methylcellulose, and water-insoluble polymers such as cross-linked carboxyl-containing polymers.

The composition can be an in situ gellable aqueous solution, suspension or solution/suspension having excipients substantially as disclosed in U.S. Pat. No. 5,192,535, comprising about 0.1% to about 6.5%, typically about 0.5% to about 4.5%, by weight, based on the total weight of the composition, of one or more cross-linked carboxyl-containing polymers. Such an aqueous suspension is typically sterile and has an osmolality of about 10 to about 400 mOsM, typically about 100 to about 250 mOsM, a pH of about 3 to about 6.5, typically about 4 to about 6, and an initial viscosity, when administered to the eye, of about 1000 to about 30,000 cPs, as measured at 25° C. using a Brookfield Digital LVT viscometer with #25 spindle and 13R small sample adapter at 12 rpm. More typically the initial viscosity is about 5000 to about 20,000 cPs. The polymer component has an average particle size not greater than about 50 μm, typically not greater than about 30 μm, more typically not greater than about 20 μm, and most typically about 1 μm to about 5 μm, in equivalent spherical diameter, and is lightly cross-linked to a degree such that, upon contact with tear fluid in the eye, which has a typical pH of about 7.2 to about 7.4, the viscosity of the suspension rapidly increases, to form a gel. This formation of a gel enables the composition to remain in the eye for a prolonged period without loss by lacrimal drainage.

Suitable carboxyl-containing polymers for use in this composition are prepared from one or more carboxyl-containing monoethylenically unsaturated monomers such as acrylic, methacrylic, ethacrylic, crotonic, angelic, tiglic, α-butylcrotonic, α-phenylacrylic, α-benzylacrylic, α-cyclohexylacrylic, cinnamic, coumaric and umbellic acids, most typically acrylic acid. The polymers are cross-linked by using less than about 5%, typically about 0.1% to about 5%, more typically about 0.2% to about 1%, by weight of one or more polyfunctional cross-linking agents such as non-polyalkenyl polyether difunctional cross-linking monomers, e.g., divinyl glycol. Other suitable cross-linking agents illustratively include 2,3-dihydroxyhexa-1,5-diene, 2,5-dimethylhexa-1,5-diene, divinylbenzene, N,N-diallylacrylamide and N,N-diallylmethacrylamide. Divinyl glycol is typically employed. Polyacrylic acid cross-linked with divinyl glycol is called polycarbophil. A polymer system containing polycarbophil is commercially available under the trademark DuraSite® of InSite Vision Inc., Alameda, Calif., as a sustained-release topical ophthalmic delivery system.

In another formulation, the composition can be an in situ gellable aqueous solution, suspension or solution/suspension having excipients substantially as disclosed in U.S. Pat. No. 4,861,760, comprising about 0.1% to about 2% by weight of a polysaccharide that gels when it contacts an aqueous medium having the ionic strength of tear fluid. One such polysaccharide is gellan gum. This composition can be prepared by a procedure substantially as disclosed in U.S. Pat. No. 4,861,760.

In yet another formulation, the composition can be an in situ gellable aqueous solution, suspension or solution/suspension having excipients substantially as disclosed in U.S. Pat. No. 5,587,175, comprising about 0.2% to about 3%, typically about 0.5% to about 1%, by weight of a gelling polysaccharide, typically selected from gellan gum, alginate gum and chitosan, and about 1% to about 50% of a water-soluble film-forming polymer, typically selected from alkylcelluloses (e.g., methylcellulose, ethylcellulose), hydroxyalkylcelluloses (e.g., hydroxyethylcellulose, hydroxypropyl methylcellulose), hyaluronic acid and salts thereof, chondroitin sulfate and salts thereof, polymers of acrylamide, acrylic acid and polycyanoacrylates, polymers of methyl methacrylate and 2-hydroxyethyl methacrylate, polydextrose, cyclodextrins, polydextrin, maltodextrin, dextran, polydextrose, gelatin, collagen, natural gums (e.g., xanthan, locust bean, acacia, tragacanth and carrageenan gums and agar), polygalacturonic acid derivatives (e.g., pectin), polyvinyl alcohol, polyvinylpyrrolidone and polyethylene glycol. The composition can optionally contain a gel-promoting counterion such as calcium in latent form, for example encapsulated in gelatin. This composition can be prepared by a procedure substantially as disclosed in U.S. Pat. No. 5,587,175.

In a further formulation, the composition can be an in situ gellable aqueous solution, suspension or solution/suspension having excipients substantially as disclosed in European Patent No. 09/424,043, comprising about 0.1% to about 5% of a carrageenan gum. In this embodiment, a carrageenan having no more than 2 sulfate groups per repeating disaccharide unit is typical, including kappa-carrageenan, having 18-25% ester sulfate by weight, iota-carrageenan, having 25-34% ester sulfate by weight, and mixtures thereof.

In still another particular formulation, the composition comprises an ophthalmically acceptable mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

In another composition, the agent is solubilized at least in part by an ophthalmically acceptable solubilizing agent. The term “solubilizing agent” generally includes agents that result in formation of a micellar solution or a true solution of the agent. Certain ophthalmically acceptable nonionic surfactants, for example polysorbate 80, can be useful as solubilizing agents, as can ophthalmically acceptable glycols, polyglycols, e.g., polyethylene glycol 400, and glycol ethers.

A class of solubilizing agents suitable for use in solution and solution/suspension compositions of the invention is the cyclodextrins. Suitable cyclodextrins can be selected from α-cyclodextrin, α-cyclodextrin, α-cyclodextrin, alkylcyclodextrins (e.g., methyl-α-cyclodextrin, dimethyl-α-cyclodextrin, diethyl-α-cyclodextrin), hydroxyalkylcyclodextrins (e.g., hydroxyethyl-α-cyclodextrin, hydroxypropyl-α-cyclodextrin), carboxyalkylcyclodextrins (e.g., carboxymethyl-α-cyclodextrin), sulfoalkylether cyclodextrins (e.g., sulfobutylether-α-cyclodextrin), and the like. Ophthalmic applications of cyclodextrins have been reviewed by Rajewski & Stella (1996), Journal of Pharmaceutical Sciences, 85, 1154, at pages 1155-1159. If desired, complexation of an agent by a cyclodextrin can be increased by addition of a water-soluble polymer such as carboxymethylcellulose, hydroxypropyl methylcellulose or polyvinylpyrrolidone, as described by Loftsson (1998), Pharmazie, 53, 733-740.

In some embodiments, one or more ophthalmically acceptable pH adjusting agents or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an ophthalmically acceptable range.

In another embodiment, one or more ophthalmically acceptable salts can be included in the composition in an amount required to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate. Optionally one or more ophthalmically acceptable acids having at least two dissociable hydrogen groups can be included in a polymer-containing composition as interactive agents to retard release of the agent through inhibition of erosion of the polymer, as disclosed in International Patent Publication No. WO 95/03784. Acids useful as interactive agents include boric, lactic, orthophosphoric, citric, oxalic, succinic, tartaric and formic glycerophosphoric acids.

In still another embodiment, an ophthalmically acceptable xanthine derivative such as caffeine, theobromine or theophylline can be included in the composition, substantially as disclosed in U.S. Pat. No. 4,559,343, to reduce ocular discomfort associated with administration of the composition.

In yet another embodiment, one or more ophthalmically acceptable preservatives can be included in the composition to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In a further embodiment, one or more ophthalmically acceptable surfactants, typically nonionic surfactants, can be included in the composition to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

In another embodiment, one or more antioxidants can be included in the composition to enhance chemical stability where required. Suitable antioxidants include ascorbic acid and sodium metabisulfite.

In still another embodiment, one or more ophthalmic lubricating agents can optionally be included in the composition to promote lacrimation or as a “dry eye” medication. Such agents include polyvinyl alcohol, methylcellulose, hydroxypropyl methylcellulose, polyvinylpyrrolidone, etc.

Aqueous suspension compositions of the invention can be packaged in single-dose non-reclosable containers. Such containers can maintain the composition in a sterile condition and thereby eliminate need for preservatives such as mercury-containing preservatives, which can sometimes cause irritation and sensitization of the eye. Alternatively, multiple-dose reclosable containers can be used, in which case it is typical to include a preservative in the composition.

As a further alternative, the composition can take the form of a solid article that can be inserted between the eye and eyelid or in the conjunctival sac, where it releases the agent as described, for example, in U.S. Pat. No. 3,863,633 and U.S. Pat. No. 3,868,445, both to Ryde & Ekstedt, incorporated herein by reference. Release is to the lacrimal fluid that bathes the surface of the cornea, or directly to the cornea itself, with which the solid article is generally in intimate contact. Solid articles suitable for implantation in the eye in such fashion are generally composed primarily of polymers and can be biodegradable or non-biodegradable. Biodegradable polymers that can be used in preparation of ocular implants carrying an AQP modulating agent or aqueous humor modulating agent in accordance with the present invention include without restriction aliphatic polyesters such as polymers and copolymers of poly(glycolide), poly(lactide), poly(α-caprolactone), poly(hydroxybutyrate) and poly(hydroxyvalerate), polyamino acids, polyorthoesters, polyanhydrides, aliphatic polycarbonates and polyether lactones. Suitable non-biodegradable polymers include silicone elastomers.

In another aspect of the invention, the composition is not administered directly to the eye. By way of example, such a composition can be administered orally, parenterally, by inhalation spray, rectally, or topically in dosage unit formulations containing conventional nontoxic pharmaceutically acceptable carriers, adjuvants, and vehicles as desired.

Solid dosage forms for oral administration can include capsules, tablets, pills, powders, and granules. In such solid dosage forms, the agents of this invention are ordinarily combined with one or more adjuvants appropriate to the indicated route of administration. If administered per os, an agent can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets and pills can additionally be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The term parenteral includes subcutaneous injections, intravenous, intramuscular, intrasternal injection, or infusion techniques. Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. A contemplated therapeutic compound can be dissolved in water, polyethylene glycol, propylene glycol, ethanol, corn oil, cottonseed oil, peanut oil, sesame oil, benzyl alcohol, sodium chloride solution, or various buffers. Other adjuvants and modes of administration are well and widely known in the pharmaceutical art.

Dosages

In general, the actual effective amounts of AQP modulating agent can and will vary according to the specific composition being utilized, the mode of administration and the age, weight and condition of the subject. Dosages for a particular individual subject can be determined by one of ordinary skill in the art using conventional considerations.

By way of example, when the AQP modulating agent is an angiotensin-converting enzyme inhibitor administered orally, suitable dosages and dosing regimens are shown in Table 1 for several inhibitors.

TABLE 1
Dosages of Oral Angiotensin-Converting Enzyme Inhibitors

| Agent | Target Dose |
|---|---|
| Benazepril (Lotensin/Novartis) | 20-40 mg QD or divided BID |
| Captopril (Capoten/Bristol-Myers Squibb) | HTN: 25-150 mg BID-TID |
| | HF: 50-100 mg TID |
| | Post-MI: 50 mg TID |
| | DN: 25 mg TID |
| Enalapril (Vasotec/Merck & Co.) | HTN: 10-40 mg QD or divided HID |
| | HF: 2.5-20 mg divided BID |
| | ALVD: 20 mg divided BID |
| Fosinopril (monopril/Bristol-Myers Squibb) | HTN, HF: 20-40 mg QD or divided BID |
| Lisinopril (Prinivil/Merck, Zestril/Zeneca) | HTN: 20-40 mg QD |
| | HF: 5-20 mg QD |
| | Post-MI: 10 mg QD |
| Moexipril (univasc/Schwarz Pharma) | 7.5-30 mg QD or divided BID |
| Quinapril (Accupril/Parke-Davis) | HTN: 20-80 mg QD or divided BID |
| | HF: 20-40 mg divided BID |
| Ramipril (Altace/Hoechst Marion Roussel) | HTN: 2.5-20 mg QD or divided BID |
| | HF: 5 mg BID |
| Trandolapril (Mavik/Knoll) | 2-4 mg QD or divided BID |

By way of further example, when the AQP modulating agent is arginine vasopressin, the amount administered daily is typically from about 0.5 to about 10 micrograms/kilogram body weight per day.

By way of still further example, when the AQP modulating agent is atrial natriuretic peptide, the amount administered daily is typically from about 0.5 to about 10 micrograms/kilogram body weight per day.

Moreover, the actual effective amounts of the aqueous humor modulating agent can and will vary according to the specific composition being utilized, the mode of administration and the age, weight and condition of the subject. Dosages for a particular individual subject can be determined by one of ordinary skill in the art using conventional considerations.

By way of example, when the aqueous humor modulating agent is a beta blocker, adrenergic agonist, CA inhibitor, cholinergic agonist, prostaglandin analog, or alpha agonist, suitable dosages and dosing regimens are shown in Table 2 for several agents belonging to each class.

TABLE 2

| Generic Name | Brand Name | Strength | Administration |
|---|---|---|---|
| Beta Blockers | | | |
| Betaxolo | Betoptic | 0.25-0.5% | BID |
| Carteolol | Ocupress | 1% | BID |
| Levobunolol | Betagan | 0.25-0.5% | QD-BID |
| Metipranolol | OptiPranolol | 0.30% | BID |
| Timolol | Timpotic/Betimol | 0.25-0.5% | BID (gel QD) |
| Levobetaxolol | Betaxon | 0.50% | BID |
| Adrenergic Agonists | | | |
| Epinephrine | Epifren | 0.1-0.2% | QD-BID |
| Dipivefrin | Propine | 0.10% | Q12h |
| Oral Carbonic Anhydrase Inhibitors | | | |
| Acetazolamide | Diamox | 250/500 mg | QID/BID for SR |
| | | (SR) | |
| Methazolamide | Neptazane | 25-100 mg | TID |
| Cholinergic Agonists | | | |
| Pilocarpine | Isopto | 0.25-10% | BID-QID |
| | Carpine/Pilocar/Pilostat | | |
| Carbachol | Isopto | 0.75-3% | TID |
| | Carbachol/carboptic | | |
| Demacarium | Humursol | 0.125-0.25% | BID |
| Echothiophate | Phospholine Iodide | 0.03-0.25% | BID |
| Iodide | | | |
| Physostigmine | Isopto Eserine | 0.25-0.5% | TID-QID |
| Topical Carbonic Anhydrase Inhibitors | | | |
| Dorzolamide | Trusopt | 2% | TID |
| Brinzolamide | Azopt | 1% | TID |
| Prostaglandin Analogs | | | |
| Latanoprost | Xalatan | 0.01% | QD (in evening) |
| Unoprostone | Rescula | 0.15% | BID |
| Bimatoprost | Lumigan | 0.03% | QD (in evening) |
| Travoprost | Travatan | 0.004% | QD (in evening) |
| Alpha Agonists | | | |
| Apraclonidine | lopidine | 0.5-1% | TID |
| Brimonidine | Alphagan | 0.20% | TID |
| Combinations | | | |
| Dorzolamide/Timolol | Cosopt | 2%/0.5% | BID |

Combination Therapies

Generally speaking, it is contemplated that the composition employed in the practice of the invention may include one or more of any of the aquaporin modulating agents detailed above in combination with one or more of any of the aqueous humor modulating agents detailed above. By way of a non limiting example, Table 3 details a number of suitable combinations that are useful in the methods and compositions of the current invention. The combination may also include an isomer, a pharmaceutically acceptable salt, ester, or prodrug of any of the aquaporin modulating agents or aqueous humor modulating agents listed in Table 3.

TABLE NO. 3

| Aquaporin Modulating Agent | Aqueous Humor Modulating Agent |
|---|---|
| carbonic anhydrase inhibitor | prostaglandin |
| carbonic anhydrase inhibitor | prostaglandin analog |
| carbonic anhydrase inhibitor | beta adrenergic antagonist |
| carbonic anhydrase inhibitor | prostaglandin FP receptor antagonist |
| carbonic anhydrase inhibitor | adrenergic agonist |
| carbonic anhydrase inhibitor | cholinergic agonist |
| carbonic anhydrase inhibitor | carbonic anhydrase inhibitor |
| angiotensin converting enzyme | prostaglandin |
| inhibitor | |
| angiotensin converting enzyme | prostaglandin analog |
| inhibitor | |
| angiotensin converting enzyme | beta adrenergic antagonist |
| inhibitor | |
| angiotensin converting enzyme | prostaglandin FP receptor antagonist |
| inhibitor | |
| angiotensin converting enzyme | adrenergic agonist |
| inhibitor | |
| angiotensin converting enzyme | cholinergic agonist |
| inhibitor | |
| angiotensin converting enzyme | carbonic anhydrase inhibitor |
| inhibitor | |
| protein kinase C activator | prostaglandin |
| protein kinase C activator | prostaglandin analog |
| protein kinase C activator | beta adrenergic antagonist |
| protein kinase C activator | prostaglandin FP receptor antagonist |
| protein kinase C activator | adrenergic agonist |
| protein kinase C activator | cholinergic agonist |
| protein kinase C activator | carbonic anhydrase inhibitor |
| protein kinase A inhibitor | prostaglandin |
| protein kinase A inhibitor | prostaglandin analog |
| protein kinase A inhibitor | beta adrenergic antagonist |
| protein kinase A inhibitor | prostaglandin FP receptor antagonist |
| protein kinase A inhibitor | adrenergic agonist |
| protein kinase A inhibitor | cholinergic agonist |
| protein kinase A inhibitor | carbonic anhydrase inhibitor |
| vasoactive peptide | prostaglandin |
| vasoactive peptide | prostaglandin analog |
| vasoactive peptide | beta adrenergic antagonist |
| vasoactive peptide | prostaglandin FP receptor antagonist |
| vasoactive peptide | adrenergic agonist |
| vasoactive peptide | cholinergic agonist |
| vasoactive peptide | carbonic anhydrase inhibitor |
| adenylate cyclase inhibitor | prostaglandin |
| adenylate cyclase inhibitor | prostaglandin analog |
| adenylate cyclase inhibitor | beta adrenergic antagonist |
| adenylate cyclase inhibitor | prostaglandin FP receptor antagonist |
| adenylate cyclase inhibitor | adrenergic agonist |
| adenylate cyclase inhibitor | cholinergic agonist |
| adenylate cyclase inhibitor | carbonic anhydrase inhibitor |
| tetraethylammonium | prostaglandin |
| tetraethylammonium | prostaglandin analog |
| tetraethylammonium | beta adrenergic antagonist |
| tetraethylammonium | prostaglandin FP receptor antagonist |
| tetraethylammonium | adrenergic agonist |
| tetraethylammonium | cholinergic agonist |
| tetraethylammonium | carbonic anhydrase inhibitor |
| colchicine | prostaglandin |
| colchicine | prostaglandin analog |
| colchicine | beta adrenergic antagonist |
| colchicine | prostaglandin FP receptor antagonist |
| colchicine | adrenergic agonist |
| colchicine | cholinergic agonist |
| colchicine | carbonic anhydrase inhibitor |
| vinca alkaloid | prostaglandin |
| vinca alkaloid | prostaglandin analog |
| vinca alkaloid | beta adrenergic antagonist |
| vinca alkaloid | prostaglandin FP receptor antagonist |
| vinca alkaloid | adrenergic agonist |
| vinca alkaloid | cholinergic agonist |
| vinca alkaloid | carbonic anhydrase inhibitor |
| rhizoxin | prostaglandin |
| rhizoxin | prostaglandin analog |
| rhizoxin | beta adrenergic antagonist |
| rhizoxin | prostaglandin FP receptor antagonist |
| rhizoxin | adrenergic agonist |
| rhizoxin | cholinergic agonist |
| rhizoxin | carbonic anhydrase inhibitor |
| estramustine | prostaglandin |
| estramustine | prostaglandin analog |
| estramustine | beta adrenergic antagonist |
| estramustine | prostaglandin FP receptor antagonist |
| estramustine | adrenergic agonist |
| estramustine | cholinergic agonist |
| estramustine | carbonic anhydrase inhibitor |
| nocodazole | prostaglandin |
| nocodazole | prostaglandin analog |
| nocodazole | beta adrenergic antagonist |
| nocodazole | prostaglandin FP receptor antagonist |
| nocodazole | adrenergic agonist |
| nocodazole | cholinergic agonist |
| nocodazole | carbonic anhydrase inhibitor |
| erbuluzole | prostaglandin |
| erbuluzole | prostaglandin analog |
| erbuluzole | beta adrenergic antagonist |
| erbuluzole | prostaglandin FP receptor antagonist |
| erbuluzole | adrenergic agonist |
| erbuluzole | cholinergic agonist |
| erbuluzole | carbonic anhydrase inhibitor |
| tubulozole | prostaglandin |
| tubulozole | prostaglandin analog |
| tubulozole | beta adrenergic antagonist |
| tubulozole | prostaglandin FP receptor antagonist |
| tubulozole | adrenergic agonist |
| tubulozole | cholinergic agonist |
| tubulozole | carbonic anhydrase inhibitor |
| cytochalasin D | prostaglandin |
| cytochalasin D | prostaglandin analog |
| cytochalasin D | beta adrenergic antagonist |
| cytochalasin D | prostaglandin FP receptor antagonist |
| cytochalasin D | adrenergic agonist |
| cytochalasin D | cholinergic agonist |
| cytochalasin D | carbonic anhydrase inhibitor |
| diacylglycerol mimic | prostaglandin |
| diacylglycerol mimic | prostaglandin analog |
| diacylglycerol mimic | beta adrenergic antagonist |
| diacylglycerol mimic | prostaglandin FP receptor antagonist |
| diacylglycerol mimic | adrenergic agonist |
| diacylglycerol mimic | cholinergic agonist |
| diacylglycerol mimic | carbonic anhydrase inhibitor |
| phorbol ester | prostaglandin |
| phorbol ester | prostaglandin analog |
| phorbol ester | beta adrenergic antagonist |
| phorbol ester | prostaglandin FP receptor antagonist |
| phorbol ester | adrenergic agonist |
| phorbol ester | cholinergic agonist |
| phorbol ester | carbonic anhydrase inhibitor |

In a further embodiment, Table 4 details a number of suitable combinations that are useful in the methods and compositions of the current invention. The combination may also include an isomer, a pharmaceutically acceptable salt, ester, or prodrug of any of the aquaporin modulating agents or aqueous humor modulating agents listed in Table 4.

TABLE 4

| Aquaporin Modulating Agent | Aqueous Humor Modulating Agent |
|---|---|
| cytochalasin D | prostaglandin A |
| cytochalasin D | prostaglandin B |
| cytochalasin D | prostaglandin D |
| cytochalasin D | prostaglandin E |
| cytochalasin D | prostaglandin F |
| cytochalasin D | latanaprost |
| cytochalasin D | bimatoprost |
| cytochalasin D | unoprostone |
| cytochalasin D | travoprost |
| cytochalasin D | betaxolol |
| cytochalasin D | carteolol |
| cytochalasin D | levobunolol |
| cytochalasin D | metipranolol |
| cytochalasin D | timolol |
| cytochalasin D | levobetaxolol |
| cytochalasin D | epinephrine |
| cytochalasin D | dipivefrin |
| cytochalasin D | pilocarpine |
| cytochalasin D | pilocarpine hydrochloride |
| cytochalasin D | carbachol |
| cytochalasin D | demacarium |
| cytochalasin D | echothiophate iodine |
| cytochalasin D | physostigmine |
| cytochalasin D | acetazolamide |
| cytochalasin D | methazolamide |
| cytochalasin D | dorzolamide |
| cytochalasin D | brinzolamide |
| acetazolamide | prostaglandin A |
| acetazolamide | prostaglandin B |
| acetazolamide | prostaglandin D |
| acetazolamide | prostaglandin E |
| acetazolamide | prostaglandin F |
| acetazolamide | latanaprost |
| acetazolamide | bimatoprost |
| acetazolamide | unoprostone |
| acetazolamide | travoprost |
| acetazolamide | betaxolol |
| acetazolamide | carteolol |
| acetazolamide | levobunolol |
| acetazolamide | metipranolol |
| acetazolamide | timolol |
| acetazolamide | levobetaxolol |
| acetazolamide | epinephrine |
| acetazolamide | dipivefrin |
| acetazolamide | pilocarpine |
| acetazolamide | pilocarpine hydrochloride |
| acetazolamide | carbachol |
| acetazolamide | demacarium |
| acetazolamide | echothiophate iodine |
| acetazolamide | physostigmine |
| acetazolamide | acetazolamide |
| acetazolamide | methazolamide |
| acetazolamide | dorzolamide |
| acetazolamide | brinzolamide |
| methazolamide | prostaglandin A |
| methazolamide | prostaglandin B |
| methazolamide | prostaglandin D |
| methazolamide | prostaglandin E |
| methazolamide | prostaglandin F |
| methazolamide | latanaprost |
| methazolamide | bimatoprost |
| methazolamide | unoprostone |
| methazolamide | travoprost |
| methazolamide | betaxolol |
| methazolamide | carteolol |
| methazolamide | levobunolol |
| methazolamide | metipranolol |
| methazolamide | timolol |
| methazolamide | levobetaxolol |
| methazolamide | epinephrine |
| methazolamide | dipivefrin |
| methazolamide | pilocarpine |
| methazolamide | pilocarpine hydrochloride |
| methazolamide | carbachol |
| methazolamide | demacarium |
| methazolamide | echothiophate iodine |
| methazolamide | physostigmine |
| methazolamide | acetazolamide |
| methazolamide | methazolamide |
| methazolamide | dorzolamide |
| methazolamide | brinzolamide |
| enalapril | prostaglandin A |
| enalapril | prostaglandin B |
| enalapril | prostaglandin D |
| enalapril | prostaglandin E |
| enalapril | prostaglandin F |
| enalapril | latanaprost |
| enalapril | bimatoprost |
| enalapril | unoprostone |
| enalapril | travoprost |
| enalapril | betaxolol |
| enalapril | carteolol |
| enalapril | levobunolol |
| enalapril | metipranolol |
| enalapril | timolol |
| enalapril | levobetaxolol |
| enalapril | epinephrine |
| enalapril | dipivefrin |
| enalapril | pilocarpine |
| enalapril | pilocarpine hydrochloride |
| enalapril | carbachol |
| enalapril | demacarium |
| enalapril | echothiophate iodine |
| enalapril | physostigmine |
| enalapril | acetazolamide |
| enalapril | methazolamide |
| enalapril | dorzolamide |
| enalapril | brinzolamide |
| benazepril | prostaglandin A |
| benazepril | prostaglandin B |
| benazepril | prostaglandin D |
| benazepril | prostaglandin E |
| benazepril | prostaglandin F |
| benazepril | latanaprost |
| benazepril | bimatoprost |
| benazepril | unoprostone |
| benazepril | travoprost |
| benazepril | betaxolol |
| benazepril | carteolol |
| benazepril | levobunolol |
| benazepril | metipranolol |
| benazepril | timolol |
| benazepril | levobetaxolol |
| benazepril | epinephrine |
| benazepril | dipivefrin |
| benazepril | pilocarpine |
| benazepril | pilocarpine hydrochloride |
| benazepril | carbachol |
| benazepril | demacarium |
| benazepril | echothiophate iodine |
| benazepril | physostigmine |
| benazepril | acetazolamide |
| benazepril | methazolamide |
| benazepril | dorzolamide |
| benazepril | brinzolamide |
| captopril | prostaglandin A |
| captopril | prostaglandin B |
| captopril | prostaglandin D |
| captopril | prostaglandin E |
| captopril | prostaglandin F |
| captopril | latanaprost |
| captopril | bimatoprost |
| captopril | unoprostone |
| captopril | travoprost |
| captopril | betaxolol |
| captopril | carteolol |
| captopril | levobunolol |
| captopril | metipranolol |
| captopril | timolol |
| captopril | levobetaxolol |
| captopril | epinephrine |
| captopril | dipivefrin |
| captopril | pilocarpine |
| captopril | pilocarpine hydrochloride |
| captopril | carbachol |
| captopril | demacarium |
| captopril | echothiophate iodine |
| captopril | physostigmine |
| captopril | acetazolamide |
| captopril | methazolamide |
| captopril | dorzolamide |
| captopril | brinzolamide |
| fosinopril | prostaglandin A |
| fosinopril | prostaglandin B |
| fosinopril | prostaglandin D |
| fosinopril | prostaglandin E |
| fosinopril | prostaglandin F |
| fosinopril | latanaprost |
| fosinopril | bimatoprost |
| fosinopril | unoprostone |
| fosinopril | travoprost |
| fosinopril | betaxolol |
| fosinopril | carteolol |
| fosinopril | levobunolol |
| fosinopril | metipranolol |
| fosinopril | timolol |
| fosinopril | levobetaxolol |
| fosinopril | epinephrine |
| fosinopril | dipivefrin |
| fosinopril | pilocarpine |
| fosinopril | pilocarpine hydrochloride |
| fosinopril | carbachol |
| fosinopril | demacarium |
| fosinopril | echothiophate iodine |
| fosinopril | physostigmine |
| fosinopril | acetazolamide |
| fosinopril | methazolamide |
| fosinopril | dorzolamide |
| fosinopril | brinzolamide |
| lisinopril | prostaglandin A |
| lisinopril | prostaglandin B |
| lisinopril | prostaglandin D |
| lisinopril | prostaglandin E |
| lisinopril | prostaglandin F |
| lisinopril | latanaprost |
| lisinopril | bimatoprost |
| lisinopril | unoprostone |
| lisinopril | travoprost |
| lisinopril | betaxolol |
| lisinopril | carteolol |
| lisinopril | levobunolol |
| lisinopril | metipranolol |
| lisinopril | timolol |
| lisinopril | levobetaxolol |
| lisinopril | epinephrine |
| lisinopril | dipivefrin |
| lisinopril | pilocarpine |
| lisinopril | pilocarpine hydrochloride |
| lisinopril | carbachol |
| lisinopril | demacarium |
| lisinopril | echothiophate iodine |
| lisinopril | physostigmine |
| lisinopril | acetazolamide |
| lisinopril | methazolamide |
| lisinopril | dorzolamide |
| lisinopril | brinzolamide |
| moexipril | prostaglandin A |
| moexipril | prostaglandin B |
| moexipril | prostaglandin D |
| moexipril | prostaglandin E |
| moexipril | prostaglandin F |
| moexipril | latanaprost |
| moexipril | bimatoprost |
| moexipril | unoprostone |
| moexipril | travoprost |
| moexipril | betaxolol |
| moexipril | carteolol |
| moexipril | levobunolol |
| moexipril | metipranolol |
| moexipril | timolol |
| moexipril | levobetaxolol |
| moexipril | epinephrine |
| moexipril | dipivefrin |
| moexipril | pilocarpine |
| moexipril | pilocarpine hydrochloride |
| moexipril | carbachol |
| moexipril | demacarium |
| moexipril | echothiophate iodine |
| moexipril | physostigmine |
| moexipril | acetazolamide |
| moexipril | methazolamide |
| moexipril | dorzolamide |
| moexipril | brinzolamide |
| quinapril | prostaglandin A |
| quinapril | prostaglandin B |
| quinapril | prostaglandin D |
| quinapril | prostaglandin E |
| quinapril | prostaglandin F |
| quinapril | latanaprost |
| quinapril | bimatoprost |
| quinapril | unoprostone |
| quinapril | travoprost |
| quinapril | betaxolol |
| quinapril | carteolol |
| quinapril | levobunolol |
| quinapril | metipranolol |
| quinapril | timolol |
| quinapril | levobetaxolol |
| quinapril | epinephrine |
| quinapril | dipivefrin |
| quinapril | pilocarpine |
| quinapril | pilocarpine hydrochloride |
| quinapril | carbachol |
| quinapril | demacarium |
| quinapril | echothiophate iodine |
| quinapril | physostigmine |
| quinapril | acetazolamide |
| quinapril | methazolamide |
| quinapril | dorzolamide |
| quinapril | brinzolamide |
| ramipril | prostaglandin A |
| ramipril | prostaglandin B |
| ramipril | prostaglandin D |
| ramipril | prostaglandin E |
| ramipril | prostaglandin F |
| ramipril | latanaprost |
| ramipril | bimatoprost |
| ramipril | unoprostone |
| ramipril | travoprost |
| ramipril | betaxolol |
| ramipril | carteolol |
| ramipril | levobunolol |
| ramipril | metipranolol |
| ramipril | timolol |
| ramipril | levobetaxolol |
| ramipril | epinephrine |
| ramipril | dipivefrin |
| ramipril | pilocarpine |
| ramipril | pilocarpine hydrochloride |
| ramipril | carbachol |
| ramipril | demacarium |
| ramipril | echothiophate iodine |
| ramipril | physostigmine |
| ramipril | acetazolamide |
| ramipril | methazolamide |
| ramipril | dorzolamide |
| ramipril | brinzolamide |
| tandolapril | prostaglandin A |
| tandolapril | prostaglandin B |
| tandolapril | prostaglandin D |
| tandolapril | prostaglandin E |
| tandolapril | prostaglandin F |
| tandolapril | latanaprost |
| tandolapril | bimatoprost |
| tandolapril | unoprostone |
| tandolapril | travoprost |
| tandolapril | betaxolol |
| tandolapril | carteolol |
| tandolapril | levobunolol |
| tandolapril | metipranolol |
| tandolapril | timolol |
| tandolapril | levobetaxolol |
| tandolapril | epinephrine |
| tandolapril | dipivefrin |
| tandolapril | pilocarpine |
| tandolapril | pilocarpine hydrochloride |
| tandolapril | carbachol |
| tandolapril | demacarium |
| tandolapril | echothiophate iodine |
| tandolapril | physostigmine |
| tandolapril | acetazolamide |
| tandolapril | methazolamide |
| tandolapril | dorzolamide |
| tandolapril | brinzolamide |
| phorbol ester | prostaglandin A |
| phorbol ester | prostaglandin B |
| phorbol ester | prostaglandin D |
| phorbol ester | prostaglandin E |
| phorbol ester | prostaglandin F |
| phorbol ester | latanaprost |
| phorbol ester | bimatoprost |
| phorbol ester | unoprostone |
| phorbol ester | travoprost |
| phorbol ester | betaxolol |
| phorbol ester | carteolol |
| phorbol ester | levobunolol |
| phorbol ester | metipranolol |
| phorbol ester | timolol |
| phorbol ester | levobetaxolol |
| phorbol ester | epinephrine |
| phorbol ester | dipivefrin |
| phorbol ester | pilocarpine |
| phorbol ester | pilocarpine hydrochloride |
| phorbol ester | carbachol |
| phorbol ester | demacarium |
| phorbol ester | echothiophate iodine |
| phorbol ester | physostigmine |
| phorbol ester | acetazolamide |
| phorbol ester | methazolamide |
| phorbol ester | dorzolamide |
| phorbol ester | brinzolamide |
| phorbol 12, 13-dibutyrate | prostaglandin A |
| phorbol 12, 13-dibutyrate | prostaglandin B |
| phorbol 12, 13-dibutyrate | prostaglandin D |
| phorbol 12, 13-dibutyrate | prostaglandin E |
| phorbol 12, 13-dibutyrate | prostaglandin F |
| phorbol 12, 13-dibutyrate | latanaprost |
| phorbol 12, 13-dibutyrate | bimatoprost |
| phorbol 12, 13-dibutyrate | unoprostone |
| phorbol 12, 13-dibutyrate | travoprost |
| phorbol 12, 13-dibutyrate | betaxolol |
| phorbol 12, 13-dibutyrate | carteolol |
| phorbol 12, 13-dibutyrate | levobunolol |
| phorbol 12, 13-dibutyrate | metipranolol |
| phorbol 12, 13-dibutyrate | timolol |
| phorbol 12, 13-dibutyrate | levobetaxolol |
| phorbol 12, 13-dibutyrate | epinephrine |
| phorbol 12, 13-dibutyrate | dipivefrin |
| phorbol 12, 13-dibutyrate | pilocarpine |
| phorbol 12, 13-dibutyrate | pilocarpine hydrochloride |
| phorbol 12, 13-dibutyrate | carbachol |
| phorbol 12, 13-dibutyrate | demacarium |
| phorbol 12, 13-dibutyrate | echothiophate iodine |
| phorbol 12, 13-dibutyrate | physostigmine |
| phorbol 12, 13-dibutyrate | acetazolamide |
| phorbol 12, 13-dibutyrate | methazolamide |
| phorbol 12, 13-dibutyrate | dorzolamide |
| phorbol 12, 13-dibutyrate | brinzolamide |
| phorbol 12-myristate-12-acetate | prostaglandin A |
| phorbol 12-myristate-12-acetate | prostaglandin B |
| phorbol 12-myristate-12-acetate | prostaglandin D |
| phorbol 12-myristate-12-acetate | prostaglandin E |
| phorbol 12-myristate-12-acetate | prostaglandin F |
| phorbol 12-myristate-12-acetate | latanaprost |
| phorbol 12-myristate-12-acetate | bimatoprost |
| phorbol 12-myristate-12-acetate | unoprostone |
| phorbol 12-myristate-12-acetate | travoprost |
| phorbol 12-myristate-12-acetate | betaxolol |
| phorbol 12-myristate-12-acetate | carteolol |
| phorbol 12-myristate-12-acetate | levobunolol |
| phorbol 12-myristate-12-acetate | metipranolol |
| phorbol 12-myristate-12-acetate | timolol |
| phorbol 12-myristate-12-acetate | levobetaxolol |
| phorbol 12-myristate-12-acetate | epinephrine |
| phorbol 12-myristate-12-acetate | dipivefrin |
| phorbol 12-myristate-12-acetate | pilocarpine |
| phorbol 12-myristate-12-acetate | pilocarpine hydrochloride |
| phorbol 12-myristate-12-acetate | carbachol |
| phorbol 12-myristate-12-acetate | demacarium |
| phorbol 12-myristate-12-acetate | echothiophate iodine |
| phorbol 12-myristate-12-acetate | physostigmine |
| phorbol 12-myristate-12-acetate | acetazolamide |
| phorbol 12-myristate-12-acetate | methazolamide |
| phorbol 12-myristate-12-acetate | dorzolamide |
| phorbol 12-myristate-12-acetate | brinzolamide |
| phorbol 12-O-tetradecanoylphorbol- | prostaglandin A |
| 13-acetate | |
| phorbol 12-O-tetradecanoylphorbol- | prostaglandin B |
| 13-acetate | |
| phorbol 12-O-tetradecanoylphorbol- | prostaglandin D |
| 13-acetate | |
| phorbol 12-O-tetradecanoylphorbol- | prostaglandin E |
| 13-acetate | |
| phorbol 12-O-tetradecanoylphorbol- | prostaglandin F |
| 13-acetate | |
| phorbol 12-O-tetradecanoylphorbol- | latanaprost |
| 13-acetate | |
| phorbol 12-O-tetradecanoylphorbol- | bimatoprost |
| 13-acetate | |
| phorbol 12-O-tetradecanoylphorbol- | unoprostone |
| 13-acetate | |
| phorbol 12-O-tetradecanoylphorbol- | travoprost |
| 13-acetate | |
| phorbol 12-O-tetradecanoylphorbol- | betaxolol |
| 13-acetate | |
| phorbol 12-O-tetradecanoylphorbol- | carteolol |
| 13-acetate | |
| phorbol 12-O-tetradecanoylphorbol- | levobunolol |
| 13-acetate | |
| phorbol 12-O-tetradecanoylphorbol- | metipranolol |
| 13-acetate | |
| phorbol 12-O-tetradecanoylphorbol- | timolol |
| 13-acetate | |
| phorbol 12-O-tetradecanoylphorbol- | levobetaxolol |
| 13-acetate | |
| phorbol 12-O-tetradecanoylphorbol- | epinephrine |
| 13-acetate | |
| phorbol 12-O-tetradecanoylphorbol- | dipivefrin |
| 13-acetate | |
| phorbol 12-O-tetradecanoylphorbol- | pilocarpine |
| 13-acetate | |
| phorbol 12-O-tetradecanoylphorbol- | pilocarpine hydrochloride |
| 13-acetate | |
| phorbol 12-O-tetradecanoylphorbol- | carbachol |
| 13-acetate | |
| phorbol 12-O-tetradecanoylphorbol- | demacarium |
| 13-acetate | |
| phorbol 12-O-tetradecanoylphorbol- | echothiophate iodine |
| 13-acetate | |
| phorbol 12-O-tetradecanoylphorbol- | physostigmine |
| 13-acetate | |
| phorbol 12-O-tetradecanoylphorbol- | acetazolamide |
| 13-acetate | |
| phorbol 12-O-tetradecanoylphorbol- | methazolamide |
| 13-acetate | |
| phorbol 12-O-tetradecanoylphorbol- | dorzolamide |
| 13-acetate | |
| phorbol 12-O-tetradecanoylphorbol- | brinzolamide |
| 13-acetate | |
| phorbol 12, 13-didecanoate | prostaglandin A |
| phorbol 12, 13-didecanoate | prostaglandin B |
| phorbol 12, 13-didecanoate | prostaglandin D |
| phorbol 12, 13-didecanoate | prostaglandin E |
| phorbol 12, 13-didecanoate | prostaglandin F |
| phorbol 12, 13-didecanoate | latanaprost |
| phorbol 12, 13-didecanoate | bimatoprost |
| phorbol 12, 13-didecanoate | unoprostone |
| phorbol 12, 13-didecanoate | travoprost |
| phorbol 12, 13-didecanoate | betaxolol |
| phorbol 12, 13-didecanoate | carteolol |
| phorbol 12, 13-didecanoate | levobunolol |
| phorbol 12, 13-didecanoate | metipranolol |
| phorbol 12, 13-didecanoate | timolol |
| phorbol 12, 13-didecanoate | levobetaxolol |
| phorbol 12, 13-didecanoate | epinephrine |
| phorbol 12, 13-didecanoate | dipivefrin |
| phorbol 12, 13-didecanoate | pilocarpine |
| phorbol 12, 13-didecanoate | pilocarpine hydrochloride |
| phorbol 12, 13-didecanoate | carbachol |
| phorbol 12, 13-didecanoate | demacarium |
| phorbol 12, 13-didecanoate | echothiophate iodine |
| phorbol 12, 13-didecanoate | physostigmine |
| phorbol 12, 13-didecanoate | acetazolamide |
| phorbol 12, 13-didecanoate | methazolamide |
| phorbol 12, 13-didecanoate | dorzolamide |
| phorbol 12, 13-didecanoate | brinzolamide |
| tetradecanoylphorbol acetate | prostaglandin A |
| tetradecanoylphorbol acetate | prostaglandin B |
| tetradecanoylphorbol acetate | prostaglandin D |
| tetradecanoylphorbol acetate | prostaglandin E |
| tetradecanoylphorbol acetate | prostaglandin F |
| tetradecanoylphorbol acetate | latanaprost |
| tetradecanoylphorbol acetate | bimatoprost |
| tetradecanoylphorbol acetate | unoprostone |
| tetradecanoylphorbol acetate | travoprost |
| tetradecanoylphorbol acetate | betaxolol |
| tetradecanoylphorbol acetate | carteolol |
| tetradecanoylphorbol acetate | levobunolol |
| tetradecanoylphorbol acetate | metipranolol |
| tetradecanoylphorbol acetate | timolol |
| tetradecanoylphorbol acetate | levobetaxolol |
| tetradecanoylphorbol acetate | epinephrine |
| tetradecanoylphorbol acetate | dipivefrin |
| tetradecanoylphorbol acetate | pilocarpine |
| tetradecanoylphorbol acetate | pilocarpine hydrochloride |
| tetradecanoylphorbol acetate | carbachol |
| tetradecanoylphorbol acetate | demacarium |
| tetradecanoylphorbol acetate | echothiophate iodine |
| tetradecanoylphorbol acetate | physostigmine |
| tetradecanoylphorbol acetate | acetazolamide |
| tetradecanoylphorbol acetate | methazolamide |
| tetradecanoylphorbol acetate | dorzolamide |
| tetradecanoylphorbol acetate | brinzolamide |
| ionomycin | prostaglandin A |
| ionomycin | prostaglandin B |
| ionomycin | prostaglandin D |
| ionomycin | prostaglandin E |
| ionomycin | prostaglandin F |
| ionomycin | latanaprost |
| ionomycin | bimatoprost |
| ionomycin | unoprostone |
| ionomycin | travoprost |
| ionomycin | betaxolol |
| ionomycin | carteolol |
| ionomycin | levobunolol |
| ionomycin | metipranolol |
| ionomycin | timolol |
| ionomycin | levobetaxolol |
| ionomycin | epinephrine |
| ionomycin | dipivefrin |
| ionomycin | pilocarpine |
| ionomycin | pilocarpine hydrochloride |
| ionomycin | carbachol |
| ionomycin | demacarium |
| ionomycin | echothiophate iodine |
| ionomycin | physostigmine |
| ionomycin | acetazolamide |
| ionomycin | methazolamide |
| ionomycin | dorzolamide |
| ionomycin | brinzolamide |
| vasopressin | prostaglandin A |
| vasopressin | prostaglandin B |
| vasopressin | prostaglandin D |
| vasopressin | prostaglandin E |
| vasopressin | prostaglandin F |
| vasopressin | latanaprost |
| vasopressin | bimatoprost |
| vasopressin | unoprostone |
| vasopressin | travoprost |
| vasopressin | betaxolol |
| vasopressin | carteolol |
| vasopressin | levobunolol |
| vasopressin | metipranolol |
| vasopressin | timolol |
| vasopressin | levobetaxolol |
| vasopressin | epinephrine |
| vasopressin | dipivefrin |
| vasopressin | pilocarpine |
| vasopressin | pilocarpine hydrochloride |
| vasopressin | carbachol |
| vasopressin | demacarium |
| vasopressin | echothiophate iodine |
| vasopressin | physostigmine |
| vasopressin | acetazolamide |
| vasopressin | methazolamide |
| vasopressin | dorzolamide |
| vasopressin | brinzolamide |
| arginine vasopressin | prostaglandin A |
| arginine vasopressin | prostaglandin B |
| arginine vasopressin | prostaglandin D |
| arginine vasopressin | prostaglandin E |
| arginine vasopressin | prostaglandin F |
| arginine vasopressin | latanaprost |
| arginine vasopressin | bimatoprost |
| arginine vasopressin | unoprostone |
| arginine vasopressin | travoprost |
| arginine vasopressin | betaxolol |
| arginine vasopressin | carteolol |
| arginine vasopressin | levobunolol |
| arginine vasopressin | metipranolol |
| arginine vasopressin | timolol |
| arginine vasopressin | levobetaxolol |
| arginine vasopressin | epinephrine |
| arginine vasopressin | dipivefrin |
| arginine vasopressin | pilocarpine |
| arginine vasopressin | pilocarpine hydrochloride |
| arginine vasopressin | carbachol |
| arginine vasopressin | demacarium |
| arginine vasopressin | echothiophate iodine |
| arginine vasopressin | physostigmine |
| arginine vasopressin | acetazolamide |
| arginine vasopressin | methazolamide |
| arginine vasopressin | dorzolamide |
| arginine vasopressin | brinzolamide |
| atrial natriuretic peptide | prostaglandin A |
| atrial natriuretic peptide | prostaglandin B |
| atrial natriuretic peptide | prostaglandin D |
| atrial natriuretic peptide | prostaglandin E |
| atrial natriuretic peptide | prostaglandin F |
| atrial natriuretic peptide | latanaprost |
| atrial natriuretic peptide | bimatoprost |
| atrial natriuretic peptide | unoprostone |
| atrial natriuretic peptide | travoprost |
| atrial natriuretic peptide | betaxolol |
| atrial natriuretic peptide | carteolol |
| atrial natriuretic peptide | levobunolol |
| atrial natriuretic peptide | metipranolol |
| atrial natriuretic peptide | timolol |
| atrial natriuretic peptide | levobetaxolol |
| atrial natriuretic peptide | epinephrine |
| atrial natriuretic peptide | dipivefrin |
| atrial natriuretic peptide | pilocarpine |
| atrial natriuretic peptide | pilocarpine hydrochloride |
| atrial natriuretic peptide | carbachol |
| atrial natriuretic peptide | demacarium |
| atrial natriuretic peptide | echothiophate iodine |
| atrial natriuretic peptide | physostigmine |
| atrial natriuretic peptide | acetazolamide |
| atrial natriuretic peptide | methazolamide |
| atrial natriuretic peptide | dorzolamide |
| atrial natriuretic peptide | brinzolamide |
| brain natriuretic peptide | prostaglandin A |
| brain natriuretic peptide | prostaglandin B |
| brain natriuretic peptide | prostaglandin D |
| brain natriuretic peptide | prostaglandin E |
| brain natriuretic peptide | prostaglandin F |
| brain natriuretic peptide | latanaprost |
| brain natriuretic peptide | bimatoprost |
| brain natriuretic peptide | unoprostone |
| brain natriuretic peptide | travoprost |
| brain natriuretic peptide | betaxolol |
| brain natriuretic peptide | carteolol |
| brain natriuretic peptide | levobunolol |
| brain natriuretic peptide | metipranolol |
| brain natriuretic peptide | timolol |
| brain natriuretic peptide | levobetaxolol |
| brain natriuretic peptide | epinephrine |
| brain natriuretic peptide | dipivefrin |
| brain natriuretic peptide | pilocarpine |
| brain natriuretic peptide | pilocarpine hydrochloride |
| brain natriuretic peptide | carbachol |
| brain natriuretic peptide | demacarium |
| brain natriuretic peptide | echothiophate iodine |
| brain natriuretic peptide | physostigmine |
| brain natriuretic peptide | acetazolamide |
| brain natriuretic peptide | methazolamide |
| brain natriuretic peptide | dorzolamide |
| brain natriuretic peptide | brinzolamide |
| tetraethyl ammonium | prostaglandin A |
| tetraethyl ammonium | prostaglandin B |
| tetraethyl ammonium | prostaglandin D |
| tetraethyl ammonium | prostaglandin E |
| tetraethyl ammonium | prostaglandin F |
| tetraethyl ammonium | latanaprost |
| tetraethyl ammonium | bimatoprost |
| tetraethyl ammonium | unoprostone |
| tetraethyl ammonium | travoprost |
| tetraethyl ammonium | betaxolol |
| tetraethyl ammonium | carteolol |
| tetraethyl ammonium | levobunolol |
| tetraethyl ammonium | metipranolol |
| tetraethyl ammonium | timolol |
| tetraethyl ammonium | levobetaxolol |
| tetraethyl ammonium | epinephrine |
| tetraethyl ammonium | dipivefrin |
| tetraethyl ammonium | pilocarpine |
| tetraethyl ammonium | pilocarpine hydrochloride |
| tetraethyl ammonium | carbachol |
| tetraethyl ammonium | demacarium |
| tetraethyl ammonium | echothiophate iodine |
| tetraethyl ammonium | physostigmine |
| tetraethyl ammonium | acetazolamide |
| tetraethyl ammonium | methazolamide |
| tetraethyl ammonium | dorzolamide |
| tetraethyl ammonium | brinzolamide |
| colchicine | prostaglandin A |
| colchicine | prostaglandin B |
| colchicine | prostaglandin D |
| colchicine | prostaglandin E |
| colchicine | prostaglandin F |
| colchicine | latanaprost |
| colchicine | bimatoprost |
| colchicine | unoprostone |
| colchicine | travoprost |
| colchicine | betaxolol |
| colchicine | carteolol |
| colchicine | levobunolol |
| colchicine | metipranolol |
| colchicine | timolol |
| colchicine | levobetaxolol |
| colchicine | epinephrine |
| colchicine | dipivefrin |
| colchicine | pilocarpine |
| colchicine | pilocarpine hydrochloride |
| colchicine | carbachol |
| colchicine | demacarium |
| colchicine | echothiophate iodine |
| colchicine | physostigmine |
| colchicine | acetazolamide |
| colchicine | methazolamide |
| colchicine | dorzolamide |
| colchicine | brinzolamide |
| vinca alkaloid | prostaglandin A |
| vinca alkaloid | prostaglandin B |
| vinca alkaloid | prostaglandin D |
| vinca alkaloid | prostaglandin E |
| vinca alkaloid | prostaglandin F |
| vinca alkaloid | latanaprost |
| vinca alkaloid | bimatoprost |
| vinca alkaloid | unoprostone |
| vinca alkaloid | travoprost |
| vinca alkaloid | betaxolol |
| vinca alkaloid | carteolol |
| vinca alkaloid | levobunolol |
| vinca alkaloid | metipranolol |
| vinca alkaloid | timolol |
| vinca alkaloid | levobetaxolol |
| vinca alkaloid | epinephrine |
| vinca alkaloid | dipivefrin |
| vinca alkaloid | pilocarpine |
| vinca alkaloid | pilocarpine hydrochloride |
| vinca alkaloid | carbachol |
| vinca alkaloid | demacarium |
| vinca alkaloid | echothiophate iodine |
| vinca alkaloid | physostigmine |
| vinca alkaloid | acetazolamide |
| vinca alkaloid | methazolamide |
| vinca alkaloid | dorzolamide |
| vinca alkaloid | brinzolamide |
| rhizoxin | prostaglandin A |
| rhizoxin | prostaglandin B |
| rhizoxin | prostaglandin D |
| rhizoxin | prostaglandin E |
| rhizoxin | prostaglandin F |
| rhizoxin | latanaprost |
| rhizoxin | bimatoprost |
| rhizoxin | unoprostone |
| rhizoxin | travoprost |
| rhizoxin | betaxolol |
| rhizoxin | carteolol |
| rhizoxin | levobunolol |
| rhizoxin | metipranolol |
| rhizoxin | timolol |
| rhizoxin | levobetaxolol |
| rhizoxin | epinephrine |
| rhizoxin | dipivefrin |
| rhizoxin | pilocarpine |
| rhizoxin | pilocarpine hydrochloride |
| rhizoxin | carbachol |
| rhizoxin | demacarium |
| rhizoxin | echothiophate iodine |
| rhizoxin | physostigmine |
| rhizoxin | acetazolamide |
| rhizoxin | methazolamide |
| rhizoxin | dorzolamide |
| rhizoxin | brinzolamide |
| estramustine | prostaglandin A |
| estramustine | prostaglandin B |
| estramustine | prostaglandin D |
| estramustine | prostaglandin E |
| estramustine | prostaglandin F |
| estramustine | latanaprost |
| estramustine | bimatoprost |
| estramustine | unoprostone |
| estramustine | travoprost |
| estramustine | betaxolol |
| estramustine | carteolol |
| estramustine | levobunolol |
| estramustine | metipranolol |
| estramustine | timolol |
| estramustine | levobetaxolol |
| estramustine | epinephrine |
| estramustine | dipivefrin |
| estramustine | pilocarpine |
| estramustine | pilocarpine hydrochloride |
| estramustine | carbachol |
| estramustine | demacarium |
| estramustine | echothiophate iodine |
| estramustine | physostigmine |
| estramustine | acetazolamide |
| estramustine | methazolamide |
| estramustine | dorzolamide |
| estramustine | brinzolamide |
| nocodazole | prostaglandin A |
| nocodazole | prostaglandin B |
| nocodazole | prostaglandin D |
| nocodazole | prostaglandin E |
| nocodazole | prostaglandin F |
| nocodazole | latanaprost |
| nocodazole | bimatoprost |
| nocodazole | unoprostone |
| nocodazole | travoprost |
| nocodazole | betaxolol |
| nocodazole | carteolol |
| nocodazole | levobunolol |
| nocodazole | metipranolol |
| nocodazole | timolol |
| nocodazole | levobetaxolol |
| nocodazole | epinephrine |
| nocodazole | dipivefrin |
| nocodazole | pilocarpine |
| nocodazole | pilocarpine hydrochloride |
| nocodazole | carbachol |
| nocodazole | demacarium |
| nocodazole | echothiophate iodine |
| nocodazole | physostigmine |
| nocodazole | acetazolamide |
| nocodazole | methazolamide |
| nocodazole | dorzolamide |
| nocodazole | brinzolamide |
| erbuluzole | prostaglandin A |
| erbuluzole | prostaglandin B |
| erbuluzole | prostaglandin D |
| erbuluzole | prostaglandin E |
| erbuluzole | prostaglandin F |
| erbuluzole | latanaprost |
| erbuluzole | bimatoprost |
| erbuluzole | unoprostone |
| erbuluzole | travoprost |
| erbuluzole | betaxolol |
| erbuluzole | carteolol |
| erbuluzole | levobunolol |
| erbuluzole | metipranolol |
| erbuluzole | timolol |
| erbuluzole | levobetaxolol |
| erbuluzole | epinephrine |
| erbuluzole | dipivefrin |
| erbuluzole | pilocarpine |
| erbuluzole | pilocarpine hydrochloride |
| erbuluzole | carbachol |
| erbuluzole | demacarium |
| erbuluzole | echothiophate iodine |
| erbuluzole | physostigmine |
| erbuluzole | acetazolamide |
| erbuluzole | methazolamide |
| erbuluzole | dorzolamide |
| erbuluzole | brinzolamide |
| tubulozole | prostaglandin A |
| tubulozole | prostaglandin B |
| tubulozole | prostaglandin D |
| tubulozole | prostaglandin E |
| tubulozole | prostaglandin F |
| tubulozole | latanaprost |
| tubulozole | bimatoprost |
| tubulozole | unoprostone |
| tubulozole | travoprost |
| tubulozole | betaxolol |
| tubulozole | carteolol |
| tubulozole | levobunolol |
| tubulozole | metipranolol |
| tubulozole | timolol |
| tubulozole | levobetaxolol |
| tubulozole | epinephrine |
| tubulozole | dipivefrin |
| tubulozole | pilocarpine |
| tubulozole | pilocarpine hydrochloride |
| tubulozole | carbachol |
| tubulozole | demacarium |
| tubulozole | echothiophate iodine |
| tubulozole | physostigmine |
| tubulozole | acetazolamide |
| tubulozole | methazolamide |
| tubulozole | dorzolamide |
| tubulozole | brinzolamide |

Diagnosis of an Elevated IOP or an Ophthalmic Disorder

One aspect of the invention encompasses diagnosing a subject in need of treatment for lowering intraocular pressure or in need of treatment for an ophthalmic disorder. A number of suitable methods for diagnosing a subject in need of treatment for lowering intraocular pressure or in need of treatment for an ophthalmic disorder may be used in the practice of the invention. While the type of test employed for diagnosis is dependent upon the subject's physical symptoms, a routine eye examine is generally performed in most embodiments. A routine eye exam usually includes measuring a subject's eye pressure with any of a number of reliable instruments known in the art, such as devices that record measurements based upon a puff of air into a subject's eye. Typically, the eye exam will also include an examination of the meshwork as well. In one embodiment, the pupils are dilated so as to allow examination of the meshwork and optic nerve. The eye exam may also consist of an examination of the optic disc, such as by using three-dimensional photography. In addition, a formal examination of the peripheral field of vision is also typically carried out with a computerized visual field machine.

Indications to be Treated

The composition comprising a therapeutically effective amount of an AQP modulating agent and a therapeutically effective amount of an aqueous humor modulating agent may be employed to treat any condition resulting from elevated IOP, low IOP or aberrant ocular water transport in a subject.

In some aspects, the invention provides a method for lowering IOP in a subject. The composition may be utilized to treat any ophthalmic disorder in a subject mediated by elevated IOP. Elevated IOP is typically a level of IOP that is harmful to the optic nerve in a particular subject and can readily be determined by a skilled artisan. The IOP may be within the normal range, particularly in patients with normal pressure glaucoma. By way of example, glaucoma is characterized by a progressive neuropathy caused in part by deleterious effects resulting from increased IOP on the optic nerve. In normal individuals, IOPs range from 12 to 20 mm Hg., averaging approximately 16 mm Hg. At higher values, for instance over 22 mm Hg, there is a risk that the eye may be affected, and if left untreated, result in the formation of glaucoma.

In one embodiment, the composition may be administered to a subject where elevated IOP or aberrant ocular water transport in a subject is a causative factor in the formation of any type of glaucoma. Several different types of glaucomas exist, each having different pathophysiologies and risk factors may be treated by administration of the composition of the invention. In terms of classification, glaucomas may first be deemed to be either “primary” or “secondary.” Primary glaucomas, result directly from anatomical and/or physiological disturbances in the flow of aqueous humor, which in turn causes IOP to rise. Secondary glaucomas occur as a sequel to ocular injury (e.g., trauma inflicted to the eye) or preexisting disease (e.g., an intraocular tumor or an enlarged cataract). Though the various secondary glaucomas have different etiologies, they are similar to the primary glaucomas in that they all produce visual loss through optic neuropathy.

The composition may be advantageously administered to a subject with any form of primary glaucoma. In one alternative of this embodiment, the primary glaucoma is open-angle glaucoma (also known as chronic or simple glaucoma). Open angle glaucoma is characterized by abnormally high resistance to fluid drainage from the eye. In another alternative of this embodiment, the primary glaucoma is angle-closure glaucoma (also known as closed-angle or narrow-angle glaucoma). Angle-closure glaucoma entails closure or blockage of the anterior chamber angle by another ocular structure (usually the iris), thereby restricting outflow of aqueous humor. In still another alternative of this embodiment, the primary glaucoma is congenital glaucoma (also known as infantile glaucoma).

In another embodiment, the composition may be advantageously administered to a subject with any form of secondary glaucoma. By way of example, the secondary glaucoma may be secondary open angle glaucoma or secondary angle closure glaucoma.

In still a further embodiment, the composition is administered to subjects that have ocular hypertension, but have not yet developed glaucoma. In this embodiment, typically the subject will have an IOP greater than about 20 mm Hg, more typically greater than 21 mm Hg and even more typically, greater than about 22 mm Hg.

In yet a further embodiment, the composition may be administered to a subject having a high risk for the development of glaucoma. In addition to subjects having elevated IOP, certain groups of subjects are at risk for developing glaucoma. These groups typically include subjects with a family history of glaucoma, persons of African descent over age 40, everyone over age 60, and diabetics. In one alternative of this embodiment, the subject also has an elevated IOP.

In another embodiment, the composition may be administered to a subject taking a particular drug known to increase the incidence of glaucoma. By way of example, the corticosteroids (e.g., prednisone, dexamethasone, and hydrocortisone) are known to induce glaucoma following both ophthalmic and systemic administration systemic administration, by increasing resistance to aqueous humor outflow through the trabecular meshwork via a mechanism somehow genetically linked to primary open angle glaucoma. In particular, dexamethasone has been associated with the most pronounced increase in intraocular pressure, and ophthalmic administration generally leads to greater increases than systemic administration.

In another aspect, the composition may be administered to a subject having an ophthalmic disorder mediated by aberrant ocular water transport. By way of example, the ophthalmic disorder may be idiopathic macular edema, corneal edema, diabetic macular edema, post-cataract macular edema, central serous retinopathy or any venous occlusive disorder of the retina.

### EXAMPLES

In the examples below, a combination therapy contains an aqueous humor modulating agent and an aquaporin modulating agent. The efficacy of such combination therapy can be evaluated in comparison to a control treatment such as a placebo treatment, administration of an aquaporin modulating agent only, or administration of an aqueous humor modulating agent only. By way of example, a combination therapy may contain a vasoactive peptide and a prostanglandin or prostaglandin analog, an angiotensin converting enzyme inhibitor and a cholinergic agonist, a protein kinase C activator and a beta adrenergic antagonist, a protein A inhibitor and carbonic anhydrase inhibitor, or a vasoactive peptide and an adrenergic agonist. It should be noted that these are only several examples, and that any of the aquaporin modulating agents and aqueous humor modulating agents detailed in the present invention, including the combinations set forth in Tables 3 or 4 may be tested as a combination therapy. The dosages of an aqueous humor modulating agent and an aquaporin modulating agent in a particular therapeutic combination may be readily determined by a skilled artisan conducting the study. The length of the study treatment will vary on a particular study and can also be determined by one of ordinary skill in the art. By way of example, the combination therapy may be administered for 12 weeks. The composition can be administered by any route as described herein, but is preferably administered as an ocular formulation directly to the eye of the subject being tested.

IOP Animal Study

The laboratory animal study can generally be performed as described in Savinova et al., BMC Genetics 2:12, Aug. 9, 2001.

Animal Husbandry

All experiments are performed in compliance with the ARVO statement for use of animals in ophthalmic and vision research. Briefly, mice are housed in cages containing white pine bedding and covered with polyester filters. For most experiments, the mice are fed NIH31 (6% fat) chow ad libitum, and their water is acidified to pH 2.8 to 3.2. The mice are housed based on the experimental group and the cages are changed one time per week. If any cage appears soiled between scheduled changes, the mice are placed in a clean cage. The environment is kept at 21° C. with a 14 hour light: 10 hour dark cycle. The colony is monitored for specific pathogens routinely.

Mice chosen for this study can be of C57BL/6J (Bl/6) strain; however, other strains can also be used. Since glaucoma, which is associated with high intraocular pressure generally occurs in older individuals, mice used herein are older, between about 12 months and 24 months of age. It should be noted that the same experiment can be performed with younger animals, if desired. Control mice are selected from the same strain and same age group as the experimental mice (receiving combination therapy). By way of example, if the experimental group comprises 10 Bl/6 mice, 3 Bl/6 mice can be used as a control.

Mice that have elevated intraocular pressure can also be used in this study. For example, mice that are heterozygous for bone morphogenetic protein 4 (Bmp4⁺/₋mice) have anterior segment abnormalities including malformed, absent or blocked trabecular meshwork and Schlemm's canal drainage structures. Mice with severe drainage structure abnormalities over 80% or more of their angle's extent have elevated IOP. The penetrance and severity of abnormalities is strongly influenced by genetic background, being most severe on the Bl/6 background. On the Bl/6 background, there is a persistence of hyaloid vasculature, diminished numbers of inner retinal cells, and absence of the optic nerve. See, e.g., Chang et al., BMC Genetics, 2:18, Nov. 6, 2001. Accordingly, an experimental group can consist of Bmp4⁺/₋mice receiving combination therapy, whereas the control group consists of Bmp4⁺/₋mice receiving a placebo treatment. The placebo treatment can be readily determined by a skilled artisan; for example, if the combination therapy is administered intravenously or intraperitoneally, the vehicle used for such administration can be used as a placebo.

Combination Treatment

Mice in the experimental group are administered the combination therapy as described above by any of the acceptable routes, e.g., intraperitoneal or intravenous. The duration and frequency of the treatment can readily be determined by a skilled artisan. By way of example, the combination therapy can be administered once a day for a period of 2 weeks. The amount of the therapy to be administered can also be readily determined by one skilled in the art. Control mice are treated according to the same protocol, except that they are administered a placebo rather than a combination therapy. Following the treatment, eyes of both the experimental and control mice are examined to determine the effect of the treatment. The result can be evaluated by determining intraocular pressure, and e.g., by performing immunohistochemistry on the eyes. For example, histochemistry (performed as described below) can be used to determine if the iridocorneal angle and aqueous humor drainage structures are open to the anterior chamber and have normal morphology.

Intraocular Pressure (IOP)

Intraocular pressure is measured as described, for example, in John S W M, Hagaman J R, MacTaggart T E, Peng L, Smithes O: Intraocular pressure in inbred mouse strains, Invest. Ophthalmol. Vis. Sci. 1997, 38:249-253. The mice are typically acclimatized to the procedure room for at least 2 weeks prior to measurement, but sometimes between 1 and 2 weeks.

All dark period measurements are made between 1 and 3 hours after the lights are turned off. The room is equipped with dim red lights and mice are protected from all light exposure during set up. Each mouse is briefly exposed to the red light when the anesthetic agents are administered. When adequate anesthesia is achieved (after 3 to 4 minutes), the mouse is placed on the measurement platform and the white light of the microscope is turned on (for approximately 1 and a half minutes) to allow ocular cannulation. The white light is used at very low intensity and is dim to minimize, if not eliminate possibility that this brief exposure alters the IOP. All other mice are protected from light exposure throughout the time an individual mouse is analyzed.

Clinical Examinations

Anterior chambers are examined with a slit lamp and photographs are taken using a 40X objective lens. An indirect ophthalmoscope and a 60 or 90 diopter lens is used to visualize the retinas and optic nerves. For this analysis, pupils are dilated with a drop of 1% cyclopentolate.

Histological Analysis

Eyes from at least several mice from the experimental and control group are fixed (4% paraformaldehyde or Fekete's acid-alcohol-formalin fixative) processed, paraffin embedded and sectioned as previously reported¹, except that the paraformaldehyde is buffered with 0.1 M phosphate buffer. A number of the eyes are processed for plastic embedding (Historesin, Leica, Heidelberg, Germany), and sectioned as previously reported². Saggital sections including the pupil and optic nerve are collected and analyzed as they contain most ocular structures. ¹Chang et al., Nat. Genet. 1999, 21:405-409 and Smith R S, Nishina P M, Ikeda S, Jewett P, Zabaleta A, John S W M: Interpretation of Ocular Pathology in Genetically-Engineered and Spontaneous Mutant Mice. In: Pathology of Genetically Engineered Mice Edited by Ward J, Sundberg J. pp. 217-231. Iowa: University of Iowa Press; 2000, 217-231 ²John et al., Invest. Ophthalmol. Vis. Sci. 1998, 39:951-962 and Smith R S, Nishina P M, Ikeda S, Jewett P, Zabaleta A, John S W M: Interpretation of Ocular Pathology in Genetically-Engineered and Spontaneous Mutant Mice. In: Pathology of Genetically Engineered Mice Edited by Ward J, Sundberg J. pp. 217-231. Iowa: University of Iowa Press; 2000, 217-231

Results

Older Bl/6 mice can be used to determine if the combination therapy provides a prophylactic or therapeutic (if the mice have a high IOP) benefit. The benefit(s) can be evaluated by determining IOP levels prior and post treatment. Furthermore, the histology can be used to evaluate the presence or absence of pathological ocular features before and after the treatment.

When Bmp4⁺/₋mice are used, it is expected that the combination therapy will result in a decrease in IOP in these mice following the treatment regimen. Eye histochemistry as described above can also be used to evaluate whether the treatment results in any improvement of drainage structure abnormalities.

It should be noted that all of the above-mentioned procedures can be modified for a particular study, depending on factors such as a drug combination used, length of the study, subjects that are selected, etc. Such modifications can be designed by a skilled artisan without undue experimentation.

What is claimed is:

1. A method of lowering intraocular pressure, the method comprising:
(a) diagnosing a subject for a condition mediated by elevated intraocular pressure; and
(b) administering to the subject an aquaporin modulating agent and an aqueous humor modulating agent, wherein the aqueous humor modulating agent lowers intraocular pressure by a pathway other than the modulation of aquaporin.

2. The method of claim 1 wherein the aquaporin modulating agent is an angiotensin converting enzyme inhibitor.

3. The method of claim 2 wherein the angiotensin converting enzyme inhibitor is selected from the group consisting of enalapril, benazepril, captopril, fosinopril, lisinopril, moexipril, quinapril, ramipril, and trandolapril.

4. The method of 1 wherein the aquaporin modulating agent is a protein kinase C activator.

5. The method of claim 4 wherein the protein kinase C activator is a diacylglycerol mimic.

6. The method of claim 5 wherein the diacylglycerol mimic is a phorbol ester.

7. The method of claim 6 wherein the phorbol ester is selected from the group consisting of phorbol 12, 13 dibutyrate, phorbol 12-myristate-12-acetate, phorbol 12-O-tetradecanoylphorbol 13-acetate, phorbol 12, 13 didecanoate and tetradecanoylphorbol acetate.

8. The method of claim 4 wherein the protein kinase C activator is ionomycin.

9. The method of claim 1 wherein the aquaporin modulating agent is a protein kinase A inhibitor.

10. The method of claim 9 wherein the protein kinase A inhibitor is selected from the group consisting of (5-isoquinolinesulfonyl)piperazine; 1-(5-Isoquinolinesulfonyl)-2-methylpiperazine, 4-cyano-3-methylisoquinoline; adenosine 3′,5′-cyclic monophosphorothioate, 2′-O-monobutyryl; adenosine 3′,5′-cyclic monophosphorothioate; 8-bromo-2′-monobutyryl, adenosine 3′,5′-cyclic monophosphorothioate; 8-piperidino, N-(2-aminoethyl)-5-chloronaphthalene-1-sulfonamide; N-(2-aminoethyl)-5-isoquinolinesulfonamide; N-[2-((p-bromocinnamyl)amino)ethyl]-5-isoquinolinesulfonamide; N-(2-guanidinoethyl)-5-isoquinolinesulfonamide; 4,4′,5,5′,6,6′-hexahydroxydiphenic acid 2,6,2′,6′-dilactone; (5-isoquinolinesulfonyl) homopiperazine; N-[2-(methylamino)ethyl]-5-isoquinolinesulfonamide; and trans-3,3′,4,5′-tetrahydroxystilbene.

11. The method of claim 1 wherein the aquaporin modulating agent is a vasoactive peptide.

12. The method of claim 11 wherein the vasoactive peptide is a vasopressin.

13. The method of claim 12 wherein the vasopressin is arginine vasopressin.

14. The method of claim 11 wherein the vasoactive peptide is atrial natriuretic peptide or brain natriuretic peptide.

15. The method of claim 1 wherein the aquaporin modulating agent is tetraethylammonium.

16. The method of claim 1 wherein the aquaporin modulating agent is colchicine.

17. The method of claim 1 wherein the aquaporin modulating agent is a vinca alkaloid.

18. The method of claim 1 wherein the aquaporin modulating agent is rhizoxin.

19. The method of claim 1 wherein the aquaporin modulating agent is estramustine.

20. The method of claim 1 wherein the aquaporin modulating agent is nocodazole.

21. The method of claim 1 wherein the aquaporin modulating agent is erbuluzole.

22. The method of claim 1 wherein the aquaporin modulating agent is tubulozole.

23. The method of claim 1 wherein the aqueous humor modulating agent is a prostaglandin or a prostaglandin analog.

24. The method of claim 23 wherein the aqueous humor modulating agent is a prostaglandin.

25. The method of claim 24 wherein the prostaglandin is selected from prostaglandin A, prostaglandin B, prostaglandin D, prostaglandin E, and prostaglandin F.

26. The method of claim 23 wherein the aqueous humor modulating agent is a prostaglandin analog.

27. The method of claim 26 wherein the prostaglandin analog is a prostaglandin. FP receptor antagonist.

28. The method of claim 26 wherein the prostaglandin analog is selected from the group consisting of latanaprost, bimatoprost, unoprostone, and travoprost.

29. The method of claim 1 wherein the aqueous humor modulating agent is a beta adrenergic antagonist.

30. The method of claim 29 wherein the beta adrenergic antagonist is selected from the group consisting of betaxolol, carteolol, levobunolol, metipranolol, timolol, and levobetaxolol.

31. The method of claim 1 wherein the aqueous humor modulating agent is an adrenergic agonist.

32. The method of claim 31 wherein the adrenergic agonist is epinephrine or dipivefrin.

33. The method of claim 1 wherein the aqueous humor modulating agent is a cholinergic agonist.

34. The method of claim 33 wherein the cholinergic agonist is selected from the group consisting of pilocarpine, pilocarpine hydrochloride, carbachol, demacarium, echothiophate idodine, and physostigmine.

35. The method of claim 1 wherein the aqueous humor modulating agent is a carbonic anhydrase inhibitor.

36. The method of claim 35 wherein the carbonic anhydrase inhibitor is selected from the group consisting of acetazolamide, methazolamide, dorzolamide hydrochloride ophthalmic solution, dorzolamide hydrochloride-timolol maleate ophthalmic solution, brinzolamide hydrochloride, dorzolamide, and brinzolamide.

37. The method of claim 1 wherein the aquaporin modulating agent is selected from the group consisting of an angiotensin converting enzyme inhibitor, a protein kinase C activator, a protein kinase A inhibitor, a vasoactive peptide, and a vinca alkaloid.

38. The method of claim 37 wherein the aqueous humor modulating agent is selected from the group consisting of a prostaglandin, a prostaglandin analog, a beta adrenergic antagonist, an adrenergic agonist, a cholinergic agonist and a carbonic anhydrase inhibitor.

39. The method of claim 1 wherein the aquaporin modulating agent is selected from the group consisting of enalapril, benazepril, captopril, fosinopril, lisinopril, moexipril, quinapril, ramipril, trandolapril, phorbol 12, 13 dibutyrate, phorbol 12-myristate-12-acetate, phorbol 12-O-tetradecanoylphorbol 13-acetate, phorbol 12, 13 didecanoate, tetradecanoylphorbol acetate, ionomycin, arginine vasopressin, atrial natriuretic peptide, brain natriuretic peptide, tetraethylammonium, colchicine, rhizoxin, estramustine, nocodazole, erbuluzole, and tubulozole.

40. The method of claim 39 wherein the aqueous humor modulating agent is selected from the group consisting of prostaglandin A, prostaglandin B, prostaglandin D, prostaglandin E, prostaglandin F, latanaprost, bimatoprost, unoprostone, travoprost, betaxolol, carteolol, levobunolol, metipranolol, timolol, levobetaxolol, epinephrine, dipivefrin, pilocarpine, pilocarpine hydrochloride, carbachol, demacarium, echothiophate iodine, physostigmine, acetazolamide, methazolamide, dorzolamide hydrochloride ophthalmic solution, dorzolamide hydrochloride-timolol maleate ophthalmic solution, brinzolamide hydrochloride, dorzolamide, and brinzolamide.

41. A method of treating an ophthalmic disorder in a subject, the method comprising:
(a) diagnosing a subject in need of treatment for an ophthalmic disorder; and
(b) administering to the subject an aquaporin modulating agent and an aqueous humor modulating agent, wherein the aqueous humor modulating agent lowers intraocular pressure by a pathway other than the modulation of aquaporin.

42. The method of claim 41 wherein the ophthalmic disorder is selected from the group consisting of idiopathic macular edema, corneal edema, diabetic macular edema, post-cataract macular edema, central serous retinopathy, venous occlusive diseases of the retina, a glaucoma disorder and ocular hypertension.

43. The method of claim 41 wherein the aquaporin modulating agent is selected from the group consisting of an angiotensin converting enzyme inhibitor, a protein kinase C activator, a protein kinase A inhibitor, a vasoactive peptide, and a vinca alkaloid.

44. The method of claim 43 wherein the aqueous humor modulating agent is selected from the group consisting of a prostaglandin, a prostaglandin analog, a beta adrenergic antagonist, an adrenergic agonist, a cholinergic agonist and a carbonic anhydrase inhibitor.

45. The method of claim 41 wherein the aquaporin modulating agent is selected from the group consisting of enalapril, benazepril, captopril, fosinopril, lisinopril, moexipril, quinapril, ramipril, trandolapril, phorbol 12, 13 dibutyrate, phorbol 12-myristate-12-acetate, phorbol 12-O-tetradecanoylphorbol 13-acetate, phorbol 12, 13 didecanoate, tetradecanoylphorbol acetate, ionomycin, arginine vasopressin, atrial natriuretic peptide, brain natriuretic peptide, tetraethylammonium, colchicine, rhizoxin, estramustine, nocodazole, erbuluzole, and tubulozole.

46. The method of claim 45 wherein the aqueous humor modulating agent is selected from the group consisting of prostaglandin A, prostaglandin B, prostaglandin D, prostaglandin E, prostaglandin F, latanaprost, bimatoprost, unoprostone, travoprost, betaxolol, carteolol, levobunolol, metipranolol, timolol, levobetaxolol, epinephrine, dipivefrin, pilocarpine, pilocarpine hydrochloride, carbachol, demacarium, echothiophate iodine, physostigmine, acetazolamide, methazolamide, dorzolamide hydrochloride ophthalmic solution, dorzolamide hydrochloride-timolol maleate ophthalmic solution, brinzolamide hydrochloride, dorzolamide, and brinzolamide.

47. A method of treating glaucoma in a subject, the method comprising:
(a) diagnosing a subject in need of treatment for glaucoma; and
(b) administering to the subject an aquaporin modulating agent and an aqueous humor modulating agent, wherein the aqueous humor modulating agent lowers intraocular pressure by a pathway other than the modulation of aquaporin.

48. The method of claim 47 wherein the glaucoma is selected from the group consisting of primary open angle glaucoma, secondary open angle glaucoma, primary angle closure glaucoma, secondary angle closure glaucoma, congenital glaucoma, and normal pressure glaucoma.

49. The method of claim 47 wherein the aquaporin modulating agent is selected from the group consisting of an angiotensin converting enzyme inhibitor, a protein kinase C activator, a protein kinase A inhibitor, a vasoactive peptide, and a vinca alkaloid.

50. The method of claim 49 wherein the aqueous humor modulating agent is selected from the group consisting of a prostaglandin, a prostaglandin analog, a beta adrenergic antagonist, an adrenergic agonist, a cholinergic agonist and a carbonic anhydrase inhibitor.

51. The method of claim 47 wherein the aquaporin modulating agent is selected from the group consisting of enalapril, benazepril, captopril, fosinopril, lisinopril, moexipril, quinapril, ramipril, trandolapril, phorbol 12, 13 dibutyrate, phorbol 12-myristate-12-acetate, phorbol 12-O-tetradecanoylphorbol 13-acetate, phorbol 12, 13 didecanoate, tetradecanoylphorbol acetate, ionomycin, arginine vasopressin, atrial natriuretic peptide, brain natriuretic peptide, tetraethylammonium, colchicine, rhizoxin, estramustine, nocodazole, erbuluzole, and tubulozole.

52. The method of claim 51 wherein the aqueous humor modulating agent is selected from the group consisting of prostaglandin A, prostaglandin B, prostaglandin D, prostaglandin E, prostaglandin F, latanaprost, bimatoprost, unoprostone, travoprost, betaxolol, carteolol, levobunolol, metipranolol, timolol, levobetaxolol, epinephrine, dipivefrin, pilocarpine, pilocarpine hydrochloride, carbachol, demacarium, echothiophate iodine, physostigmine, acetazolamide, methazolamide, dorzolamide hydrochloride ophthalmic solution, dorzolamide hydrochloride-timolol maleate ophthalmic solution, brinzolamide hydrochloride, dorzolamide, and brinzolamide.

53. The method of claim 1 wherein the subject is a human.

54. The method of claim 1 wherein the aquaporin modulating agent and the aqueous humor modulating agent are administered substantially simultaneously.

55. The method of claim 1 wherein the aquaporin modulating agent and the aqueous humor modulating agent are administered sequentially.
